(19) United States
(12) Patent Application Publication
(10) Pub. No.: US 2026/0356920 A1
(43) Pub. Date: Nov. 12, 2026

(54) A HUMAN VH-BASED SCAFFOLD FOR THE PRODUCTION SINGLE DOMAIN ANTIBODIES AND THEIR USE

(71) Applicants: THE SCRIPPS RESEARCH INSTITUTE, La Jolla, CA (US); INTERNATIONAL AIDS VACCINE INITIATIVE, New York, NY (US)

(72) Inventors: Joseph JARDINE, San Diego, CA (US); Laetitia MINDREBO, La Jolla, CA (US)

(21) Appl. No.: 19/102,044

(22) PCT Filed: Aug. 9, 2023
(86) PCT No.: PCT/US2023/071893

§ 371 Date: Feb. 7, 2025

Related U.S. Application Data
(60) Provisional application No. 63/396,782, filed on Aug. 10, 2022.

(51) Int. Cl. C07K 16/104; A01K 67/0278; C07K 16/00; C07K 16/102; G01N 33/68

(52) U.S. Cl. CPC C07K 16/104; A01K 67/0278; C07K 16/005; C07K 16/102; G01N 33/6854; A01K 2217/052; A01K 2227/105; A01K 2267/01; C07K 2317/31; C07K 2317/34; C07K 2317/52; C07K 2317/55; C07K 2317/565; C07K 2317/569; C07K 2317/624; C07K 2317/92; C07K 2319/30

(57) ABSTRACT

Provided herein are a human VH-based scaffold for the production of single domain antibodies and their use.

### CROSS-REFERENCE TO RELATED APPLICATIONS

This application claims the benefit of U.S. application No. 63/396,782, filed Aug. 10, 2022, which is incorporated herein by reference in its entirety.

### FIELD OF THE INVENTION

The field of the invention generally relates to engineered antibodies and their use.

### BACKGROUND

The use of biologics for prophylactic prevention or treatment of viral disease is increasing. Palivizumab was the first monoclonal antibody (mAb) approved for the prevention of respiratory syncytial virus (RSV) in infants, but the outbreaks of Ebola and COVID-19 have led the development of several additional products, first receiving emergency use authorization before eventually receiving full FDA approval. Antibodies (Abs) have the potential to be transformative within this space due to their high specificity, long half-life and ability to coordinate and fine-tune the response from the innate immune system via Fc-mediated interactions. One of the unique challenges in using Abs for anti-viral indications is the ever-present risk of resistant viral variants emerging, witnessed in real time during the COVID-19 epidemic as SARS-CoV-2 variants of concern (VOCs) emerged that were partially or fully resistant to the therapeutic Ab countermeasures (1). Strategies to counter this viral escape generally focus on targeting functional conserved epitopes and/or the use of multiple Abs that recognize non-overlapping epitopes, thus requiring multiple mutations for the virus to effectively evade those Abs (2). Delivering multiple Abs in a cocktail is the most straightforward solution, but the multiple biologics must now be considered during formulation and subsequent PK/PD analysis. Bi- and tri-specific Ab formats address these obstacles by creating a single molecule but require additional engineering and/or purifications to ensure that the component Abs within the multispecific maintain correct heavy/light chain pairing.

An interesting alternative biologics platform is the use of single domain “nanobodies” (Nbs). Nbs exist as a monomeric variable heavy (V_{HH}) domain only but exhibit similar antigen binding properties to conventional Abs that exist as a variable heavy (V_{H}) and variable light (V_{L}) heterodimers (3). The most common method to obtain Nbs requires immunizing camelid (e.g., llamas and alpacas) that produce V_{HH}s as part of their Ig repertoire, but phage- and yeast-based synthetic platforms can also be used for de novo discovery. To date, neutralizing Nbs have been developed for numerous viruses including HIV (4, 5), influenza (6), RSV (7, 8), SARS-CoV-2 (9, 10), hepatitis C (11) and rabies virus (12). In most instances the monomeric V_{HH} exhibits moderate neutralizing potency, but the generation of homo- or hetero-oligomers via flexible linkers and/or Fc fusion substantially improves the potency (9, 13). This modular property, in conjunction with the smaller size of the single domain molecule, allows for Nb-based molecules to target viral proteins in ways inaccessible to conventional Abs. While the value of combining multiple Nbs into a single molecule has been clearly demonstrated (6, 9, 10, 13, 14), selecting the candidate Nbs to include generally requires either a priori structural information, or the screening of many constructs to find synergistic Nb combinations.

There is a need for new human V_{H}-based scaffolds for the production of single domain antibodies.

### BRIEF SUMMARY

In one aspect, provided herein is a VH domain comprising a structure of framework 1 (FW1)-Complementarity Determining Region 1 (CDR1)-FW2-CDR2-FW3-CDR3-FW4, wherein the VH domain comprises: a) one or more of a tyrosine at position 37 (Y37), glutamate at position 44 (E44), and arginine at position 45 (R45); b) cysteines at positions 49 and 69 (C49 and C69), wherein the cysteines are capable of forming a disulfide bond; and c) one or more of a glutamine at position 5 (Q5), glycine at position 35 (G35), lysine at position 83 (K83), proline at position 84 (P84), tyrosine at position 102 (Y102) and glutamine at position 108 (Q108), wherein the amino acid positions are according to Kabat. In some embodiments, the VH domain comprises Y37, E44 and R45. In some embodiments, the VH domain comprises Q3, G35, K83, P84, Y102 and Q108. In some embodiments, the VH domain comprises Q3, G35, Y37, E44, R45, K83, P84, Y102 and Q108. In some embodiments, the CDR3 region comprises 5 to 20 amino acid residues. In some embodiments, the FW3-CDR3-FW4 portion of the VH domain comprises the amino acid sequence of 92-CARX(5 to 15)FDYW-103, wherein X(5 to 15) stands for 5 to 15 amino acid residues. In some embodiments, the VH domain is capable of specifically binding to a target of interest.

In some embodiment, a VH domain described herein comprises one or more of a) the amino acid sequence of EVQLQESGGGLVQPGGSLRLSCAASGFTFS (SEQ ID NO: 1) comprising 0, 1, 2, 3, 4 or 5 substitutions; b) the amino acid sequence of WYRQAPGKEREWVC (SEQ ID NO: 2) comprising 0, 1, 2, 3, 4 or 5 substitutions; c) the amino acid sequence of RFTCSRDNSKNTLYLQMNSLKPEDTAVYYCAX (SEQ ID NO: 3) comprising 0, 1, 2, 3, 4 or 5 substitutions; and d) the amino acid sequence of WGQGTQVTVSS (SEQ ID NO: 4) comprising 0, 1, 2, 3, 4 or 5 substitutions. In some embodiment, the VH domain comprises a) the amino acid sequence of EVQLQESGGGLVQPGGSLRLSCAASGFTFS (SEQ ID NO: 1) comprising 0, 1, 2, 3, 4 or 5 substitutions; b) the amino acid sequence of WYRQAPGKEREWVC (SEQ ID NO: 2) comprising 0, 1, 2, 3, 4 or 5 substitutions; c) the amino acid sequence of RFTCSRDNSKNTLYLQMNSLKPEDTAVYYCAX (SEQ ID NO: 3) comprising 0, 1, 2, 3, 4 or 5 substitutions; and d) the amino acid sequence of WGQGTQVTVSS (SEQ ID NO: 4) comprising 0, 1, 2, 3, 4 or 5 substitutions. In some embodiments, the VH domain comprises the amino acid sequence of SEQ ID NO: 1, 2, 3 and 4. In some embodiments, the VH domain comprises a) an amino acid sequence having at least 80%, 85%, 90%, 95%, 97%, 97%, 98% 99% or 100% identity to EVQLQESGGGLVQPGGSLRLSCAASGFTFSSYAMGWYRQAPGKEREWVCAISGSGGSTYYADS VKGRFTCSRDNSKNTLYLQMNSLKPEDTAVYYCA (SEQ ID NO: 5); and b) the amino acid sequence of WGQGTQVTVSS (SEQ ID NO: 4) comprising 0, 1, 2, 3, 4 or 5 substitutions. In some embodiments, the VH domain comprises the amino acid sequence of SEQ ID NO: 5 and 4. In some embodiments, the VH domain comprises a) an amino acid sequence having at least 80%, 85%, 90%, 95%, 97%, 97%, 98% 99% or 100% identity to EVQLQESGGGLVQPGGSLRLSCAASGFTFSSYAMGWYRQAPGKEREWVCAISGSGGSTYYADS VKGRFTCSRDNSKNTLYLQMNSLKPEDTAVYYCA (SEQ ID NO: 5); and b) the amino acid sequence of DYWGQGTQVTVSS (SEQ ID NO: 6) comprising 0, 1, 2, 3, 4 or 5 substitutions. In some embodiments, the VH domain comprises the amino acid sequence of SEQ ID NO: 5 and 6. In some embodiments, the CDR3 region comprises 5 to 20 amino acid residues. In some embodiments, the FW3-CDR3-FW4 portion of the VH domain comprises the amino acid sequence of 92-CARX(5 to 15)FDYW-103, wherein X(5 to 15) stands for 5 to 15 amino acid residues.

In some embodiments, a VH domain described herein comprises a VH3_23 human framework.

In some embodiments of a VH domain described herein, the target of interest is a pathogen derived antigen. In some embodiments, the target of interest is a pathogen derived polypeptide. In some embodiments, the pathogen derived polypeptide comprises a viral, bacterial, or parasitic polypeptide. In some embodiments, the pathogen derived polypeptide comprises a viral polypeptide.

In one aspect, provided herein is a multispecific binding agent comprising at least two VH domains described herein. In some embodiments, the at least two VH domains bind to different epitopes. In some embodiments, the at least two VH domains bind to different epitopes on the same antigen. In some embodiments, the at least two VH domains bind to different antigens. In some embodiments, the multispecific binding agent is a bispecific agent. In some embodiments, the multispecific binding agent is a trispecific agent.

In one aspect, provided herein is a fusion polypeptide comprising at least one VH domain described herein. In some embodiments, the fusion polypeptide of claim 2 comprises more than one VH domain and a heterologous polypeptide. In some embodiments, the heterologous polypeptide is a) an antibody, b) an antibody fragment, c) an Fc domain, d) transmembrane domain, or e) a membrane associating domain. In some embodiments, the heterologous polypeptide is an Fc domain.

In one aspect, provided herein is a chimeric antigen receptor (CAR) comprising at least one VH domain described herein.

In one aspect, provided herein is a host cell comprising a CAR described herein.

In one aspect, provided herein is a polynucleotide encoding the VH domain, multispecific binding agent, fusion polypeptide or CAR described herein. In some embodiments, the polynucleotide is a DNA or RNA. In some embodiments, the polynucleotide is an mRNA comprising a modification. In some embodiments, the polynucleotide is an mRNA comprising a modified nucleoside.

In one aspect, provided herein is a method of neutralizing a pathogen in a subject in need thereof, comprising administering to the subject a therapeutically effective amount of a composition comprising a VH domain described herein, or a polynucleotide encoding the VH domain.

In one aspect, provided herein is a method of treating a subject in need thereof, comprising administering to the subject a therapeutically effective amount of a composition comprising a VH domain described herein, or a polynucleotide encoding the VH domain.

In one aspect, provided herein is a VH domain library comprising a plurality of VH domains described herein.

In one aspect, provided herein is a polynucleotide library comprising a plurality of polynucleotides encoding VH domains described herein.

In one aspect, provided herein is a method for identifying a VH domain described herein capable of binding a target of interest, comprising a) contacting a VH domain library described herein with a target of interest, and b) identifying a VH domain capable of binding to the target of interest. In some embodiments, the target of interest is a pathogen derived antigen. In some embodiments, the target of interest is a pathogen derived polypeptide. In some embodiments, the pathogen derived polypeptide comprises a viral, bacterial, or parasitic polypeptide. In some embodiments, the pathogen derived polypeptide comprises a viral polypeptide.

In one aspect, provided herein is a method for identifying a polynucleotide encoding a VH domain described herein capable of binding a target of interest, comprising a) expressing a polynucleotide library described herein in a suitable host cell to produce a library of cells expressing a VH domain; b) contacting the library of cells with the target of interest, and c) identifying a polynucleotide encoding a VH domain capable of binding to the target of interest. In some embodiments, the target of interest is a pathogen derived antigen. In some embodiments, the target of interest is a pathogen derived polypeptide. In some embodiments, the pathogen derived polypeptide comprises a viral, bacterial, or parasitic polypeptide. In some embodiments, the pathogen derived polypeptide comprises a viral polypeptide.

In one aspect, provided herein is a vector comprising a) at least one functional heavy chain V gene, b) at least one heavy chain D gene and at least one heavy chain J gene, and c) a murine C gene which lacks the CH1 exon, wherein the at least one functional heavy chain V gene encodes a polypeptide comprising a fragment of the VH domain according to any one of claims 1 to 12, wherein the fragment consists of the residues corresponding to the human VH3_23 gene.

In one aspect, provided herein is a transgenic mouse host cell transformed with a vector described herein.

In one aspect, provided herein is a transgenic mouse comprising a vector described herein or a cell comprising a vector described herein.

In one aspect, provided herein is a use of the transgenic mouse described herein or the transgenic mouse host cell described herein in producing a VH only antibody or a VH domain that is capable of specifically binding to a target of interest. In some embodiments, the target of interest is a pathogen derived antigen. In some embodiments, the target of interest is a pathogen derived polypeptide. In some embodiments, the pathogen derived polypeptide comprises a viral, bacterial, or parasitic polypeptide. In some embodiments, the pathogen derived polypeptide comprises a viral polypeptide.

### BRIEF DESCRIPTION OF THE DRAWINGS

FIG. 1. A) Overlay of a VH/VL pair from a human IgG (PDB 5I19, blue) with a representative camel VHH (PDB 5U65, orange). Structures were aligned on the VHH. B) The VH domain from a human Ab that utilizes VH3-23 (PDB 5I19) with the 9 positions that are mutated to resurface the interface normally interacting with the VL (orange) in the hVHH323 scaffold and the location of the disulfide bond that was introduced through two additional Cys mutations (yellow). The variable CDR3 is colored in red. C) Deep sequencing analysis of the CDR3 loops length distribution from the transformed library compared to human (21) and camelids (22) repertoires. D) Schematic yeast display of the hVHH323 Nbs with a V5 epitope tag. E) Deep sequencing analysis of AA frequencies of diversified positions in CDR3s, showing both the intended frequencies and those observed in the transformed library.

FIG. 2. A) FACS workflow to enrich populations in antigen-specific clones. Cells were sorted with 100 nM, 20 nM and 4 nM SARS-CoV-2 RBD for AFF1, AFF2 and AFF3 steps, respectively. B) ELISA plot showing LM18-Fc, LM44-Fc, LM45-Fc and LM46-Fc binding to SARS-CoV-2 RBD. CC6.30 was used as a positive control and a Nb-Fc from the library not selected for RBD binding was used as a negative control. Assay was run in duplicate. C) Neutralization assay of Wuhan-1 SARS-CoV-2 PSV for the 4 Nbs tested showing neutralization by LM18. Assay was run in triplicate.

FIG. 3. Design of bsAbs and neutralization of Wuhan-1 SARS-CoV-2 PSV. A) FACS-based epitope binning on yeast cell surface. RBD is first added to the induced yeast cells and binds to the Nbs displayed on the surface. Then the competitive Ab (CR3022, CC12.1 or CC6.30) is incubated and binds RBD only if the Nb doesn't bind a similar epitope. B) FACS plot showing the selection of competitive (C) and non-competitive (NC) clones using an anti IgG-conjugated fluorophore. C) Nbs binning based on C/NC profile and NGS analysis. D) Confirmation of Nb epitope by BLI-based assay. One representative for the RBS-B (Nb-B) and CR3022 cryptic site (Nb-cs) class is displayed. E) Design of the bsNb4-Igs with one Nb linked to CH1 and another Nb linked to CK F) IC50 values (μM/mL) of bsNb4-Igs (Nb-B HC/LM18 LC, left and Nb-cs HC/LM18 LC, right) tested for neutralization of Wuhan-1 SARS-CoV-2 PSV. Experiments were run in duplicate. The IC50 value of LM18-Fc (2.65 μg/mL) is indicated by the purple dotted line.

FIG. 4. Neutralization of PSVs variants by bsNb4-Igs and binding kinetics. A) Neutralization of SARS-CoV-2 PSVs by LM18-based bsNb4-Igs B) Neutralization of SARS-CoV-2 PSVs by Nb-cs/Nb-B bsNb4-Igs. C) Neutralization of omicron PSV by LM18- or Nb136-based bsNb4Igs. D) Comparison between bsNb4Ig and corresponding Fabs fragments, with IC50 as molar concentration. The black and purple dotted lines indicate the absence of neutralization (≥100 μg/mL) and the IC50 value of LM18-Fc, respectively. E) SPR binding curves of LM18, Nb-B (Nb136) and Nb-cs (Nb225) for RBD and spike protein binding in different formats. In all cases, the Nb-Fc or Ab is immobilized by the Fc and the SARS-CoV-2 RBD or spike is flown as the analyte. F) Neutralization of Wuhan-1 SARS-CoV-2 PSV by LM18-based and affinity-matured LM18-based bsNb4-Igs.

FIG. 5. Structural validation of engineered Nb design. A) Our engineered Nbs showed the same backbone structure as llama Nbs. Structures of engineered Nbs (i.e. LM18, Nb136, Nb225, Nb240, and Nb255) were overlapped with VHH V, a SARS-CoV-2 Nb isolated from immunized llama (13). CDR3 of these Nbs are not considered for similarity due to high sequence diversity and are not shown. B) Cryo-EM reconstruction of LM18/Nb136 bsNb4-Ig in complex with SARS-CoV-2 6Pmut7 S protein colored by subunit/domain. C) Nb225, Nb240, and Nb255 shared the same binding mode (upper panel). Nbs-cs bind the conserved epitope site previously bound by CR3022 and COVA1-16. Coincidently, these Nbs bind SARS-CoV-2 RBD in a highly similar way as YYDRxG (SEQ ID NO: 9) Abs, i.e. COVA1-16 (lower panel). D) Cryo-EM focused classification and local refinement of a single RBD bound by LM18 and Nb136. Map shown as transparent gray, with fitted atomic models in licorice representation. E) Binding of LM18 and Nb136 with respect to the three epitopes used during FACS-based binning. Comparison of F) LM18 and G) Nb136 CDRH3 binding sites on RBD with representative neutralizing Abs.

FIG. 6. Sequence of the hNb323 scaffold (SEQ ID NO: 8). The mutated position compared to VH3 lineage are indicated in bold and blue. The positions mutated to Cys are indicated in yellow. The diversity of the CDR3 is indicated in red. Residue numbers were assigned according to the Kabat numbering system.

FIG. 7. Sampling fraction (indicated by the dotted line) of CDR3s containing 10 to 20 residues depending on the frequency. A normal distribution of the CDR3 length, such as for camelid repertoires, would result in an oversampling of the smaller CDR3 by a factor of 132 (A). A left-skewed distribution prevents such oversampling and the smaller CDR3 (10 residues) would be sampled by a factor of 5 (B).

FIG. 8. Neutralization of SARS-CoV-1 PSV by LM18-Fc and LM18.1.17-Fc. The IC50 values are 12 nM or 0.506 μg/mL and 0.9 nM or 0.037 μg/mL for LM18 and LM18.1.17, respectively. Assays were run in duplicate with a Nb starting concentration of 50 μg/mL.

FIG. 9. Evaluation of Nb-Fc by size-exclusion chromatography (SEC) using an Agilent 1260 Infinity II HPLC equipped with a TSKgel SuperSW mAb HR column (Tosoh, 7.8 mm I.D.×30 cm, 4 μm) with a 1 mL/min flow rate and detection wavelength at 280 nm. An isocratic gradient of 100% PBS was used. A) LM18; B) Nbs-B class and C) Nbs-cs class.

FIG. 10. Evaluation of Nbs and bsNb4-Igs for specific binding to Wuhan-1 SARS-CoV-2. Nbs-Fc and bsNb4-Igs were tested by ELISA for binding to Wuhan-1 SARS-CoV-2 RBD. CC6.30 was used as a positive control (green) and a Nb-Fc from the library not selected for RBD binding was used as a negative control (red). The bsNb4-Igs with LM18 either as a HC or LC were tested and no notable difference for RBD binding was observed.

FIG. 11. Evaluation of SARS-CoV-2 Nbs and IgG-like bsNb4-Igs for polyreactivity. Nbs-B and Nbs-B-based bsNb4-Igs (A) and Nbs-cs and Nb-cs-based bsNb4-Igs (B) were tested by ELISA for binding to the polyspecificity reagent (PSR): CHO-cell soluble membrane protein extracts. Bococizumab (CAS: 1407495-02-6) was used as a control (ctrl) to determine nonspecific binding to PSR. The bsNb4-Igs with LM18 either as a HC or LC were tested and no notable difference for RBD binding was observed.

FIG. 12. Epitope binning of Fc-tagged Nbs using an Octet RED384 platform. His10-tagged RBD was captured using a Ni-NTA biosensor, and indicated mAbs (CR3022, CC6.30, CC12.1) or Nb (LM18), or ACE2 at a concentration of 100 μg/ml were first incubated (*), followed by an incubation with 25 μg/ml of competing Nb (**). A) LM18; B) Nbs-B class and C) Nbs-cs class. The tested Nb # is indicated on the left of the graph.

FIG. 13. Evaluation of Nbs for specific binding to SARS-CoV-2 RBD omicron by ELISA. LM18 is shown in purple, Nbs-B in teal and Nbs-cs in salmon. Assay was run in duplicate. Nb136 (Nb-B) and Nb198, Nb225, Nb237, Nb240 and Nb255 (Nbs-cs) bind to RBD omicron, whereas LM18 and Nb121 (Nb-B) binding is highly affected by the mutations present on this variant.

FIG. 14. Neutralization of PSVs variants by Nbs and Nbs cocktails. A) Neutralization of PSVs carrying Wuhan-1 (wt) or mutated SARS-CoV-2 by Fc fused Nbs B) Neutralization of PSVs carrying Wuhan-1 (wt) or mutated SARS-CoV-2 by an equimolar ratio of by Fc fused Nbs. The purple and black dotted lines illustrate the average of LM18-Fc IC50 values and the absence of neutralization (IC50>100 μg/mL), respectively.

FIG. 15. Flow cytometry plots of the three affinity sorts for the affinity-maturation of LM18. The gates are indicated in red. Yeast display is shown on the x axis and RBD binding on the y axis.

FIG. 16. Neutralization of Wuhan-1 SARS-CoV-2 PSV by LM18-Fc and matured LM18-Fc (LM18.1.17). Assays were run in triplicate with a Nb starting concentration of 100 μg/mL.

FIG. 17. Structural details elucidated by x-ray crystallography and cryo-electron microscopy. A) Disulfide bond in the reported engineered Nbs. The blue mesh shows a 2mFc-DFo density map of the disulfide bond (shown as sticks) contoured at 1.0 σ. Nb-C4-255 is shown in light purple. SARS-CoV-2 RBD in gray. B) Sequence alignment of Nb CDR3. CDR3 of class 4 Nb225 (SEQ ID NO: 10), Nb240 (SEQ ID NO: 11), and Nb255 (SEQ ID NO: 12) showed similar binding mode as YYDRxG (SEQ ID NO: 9) antibodies, e.g. COVA1-16 (SEQ ID NO: 13) (FIG. 5) and ADI-62113 (SEQ ID NO: 14). The amino acid sequences constituting these CDR3s are aligned based on structural superposition. C) Summary schematic of focused classification and refinement methods used to generate map for LM18/Nb-C2-136 bsNb4-1g+SARS-CoV-2 6Pmut7 S model building. See Methods for additional details.

FIG. 18. IC50 values and potency for neutralization of Wuhan-1 SARS-CoV-2 PSV by 4 Nbs-Fc identified by Sanger sequencing. Assays were run in triplicate with a starting Nb concentration of 100 μg/mL.

FIG. 19. IC50 values and potency for neutralization of Wuhan-1 SARS-CoV-2 PSV by bsNb4-Igs. Assays were run in duplicate.

FIG. 20. IC50 values and potency for neutralization of SARS-CoV-2 PSVs by a selection of Nb-B/LM18 and Nb-cs/LM18 bsAbs. No notable difference for neutralization between bsNb4-Igs with LM18 either as a HC or LC could be observed. Assays were run in duplicate. wt=Wuhan-1

FIG. 21. IC50 values and potency for neutralization of SARS-CoV-2 PSVs by Nb-B/Nb-cs bsNb4-Igs. Assays were run in duplicate. wt=Wuhan-1

FIG. 22. IC50 values and potency for neutralization of SARS-CoV-2 PSVs by tetravalent monospecific Abs. Assays were run induplicate. NN: not neutralizing, n. a: not available, nt: not tested. wt=Wuhan-1

FIG. 23. Summarized results of Wuhan-1 SARS-CoV-2-RBD binding to Nbs, Fabs and Abs. Association and dissociation rate constants calculated through a 1:1 Langmuir binding model when possible or heterologous ligand binding model using the BIAevaluation software.

FIG. 24. Summarized results of Wuhan-1 SARS-CoV-2-Spike binding to Fabs or Abs. Association and dissociation rate constants calculated through a 1:1 Langmuir binding model using the BIAevaluation software.

FIG. 25. Neutralization of PSVs carrying wt and variants SARS-CoV-2 by matured LM18-based bsNb4-Igs. Assays were run in duplicate. wt=Wuhan=1

### DETAILED DESCRIPTION

In one aspect, provided herein is a VH domain comprising a structure of framework 1 (FW1)-Complementarity Determining Region 1 (CDR1)-FW2-CDR2-FW3-CDR3-FW4, wherein the VH domain comprises: a) one or more of a tyrosine at position 37 (Y37), glutamate at position 44 (E44), and arginine at position 45 (R45); b) cysteines at positions 49 and 69 (C49 and C69), wherein the cysteines are capable of forming a disulfide bond; and c) one or more of a glutamine at position 5 (Q5), glycine at position 35 (G35), lysine at position 83 (K83), proline at position 84 (P84), tyrosine at position 102 (Y102) and glutamine at position 108 (Q108), wherein the amino acid positions are according to Kabat. In some embodiments, the VH domain comprises Y37, E44 and R45. In some embodiments, the VH domain comprises Q3, G35, K83, P84, Y102 and Q108. In some embodiments, the VH domain comprises Q3, G35, Y37, E44, R45, K83, P84, Y102 and Q108. In some embodiments, the CDR3 region comprises 5 to 20 amino acid residues. In some embodiments, the FW3-CDR3-FW4 portion of the VH domain comprises the amino acid sequence of 92-CARX(5 to 15)FDYW-103, wherein X(5 to 15) stands for 5 to 15 amino acid residues. In some embodiments, the CDR3 loops all begin and end with the CAR and FDYW motifs, respectively. Between these conserved motifs, 5 to 15 randomized amino acids (AAs) are inserted, resulting in VH domains with CDR3 loop lengths of 10 to 20 AAs. In some embodiments, the VH domain is capable of specifically binding to a target of interest.

In some embodiment, a VH domain described herein comprises one or more of a) the amino acid sequence of EVQLQESGGGLVQPGGSLRLSCAASGFTFS (SEQ ID NO: 1) comprising 0, 1, 2, 3, 4 or 5 substitutions; b) the amino acid sequence of WYRQAPGKEREWVC (SEQ ID NO: 2) comprising 0, 1, 2, 3, 4 or 5 substitutions; c) the amino acid sequence of RFTCSRDNSKNTLYLQMNSLKPEDTAVYYCAX (SEQ ID NO: 3) comprising 0, 1, 2, 3, 4 or 5 substitutions; and d) the amino acid sequence of WGQGTQVTVSS (SEQ ID NO: 4) comprising 0, 1, 2, 3, 4 or 5 substitutions. In some embodiment, the VH domain comprises a) the amino acid sequence of EVQLQESGGGLVQPGGSLRLSCAASGFTFS (SEQ ID NO: 1) comprising 0, 1, 2, 3, 4 or 5 substitutions; b) the amino acid sequence of WYRQAPGKEREWVC (SEQ ID NO: 2) comprising 0, 1, 2, 3, 4 or 5 substitutions; c) the amino acid sequence of RFTCSRDNSKNTLYLQMNSLKPEDTAVYYCAX (SEQ ID NO: 3) comprising 0, 1, 2, 3, 4 or 5 substitutions; and d) the amino acid sequence of WGQGTQVTVSS (SEQ ID NO: 4) comprising 0, 1, 2, 3, 4 or 5 substitutions. In some embodiments, the VH domain comprises the amino acid sequence of SEQ ID NO: 1, 2, 3 and 4. In some embodiments, the VH domain comprises a) an amino acid sequence having at least 80%, 85%, 90%, 95%, 97%, 97%, 98% 99% or 100% identity to EVQLQESGGGLVQPGGSLRLSCAASGFTFSSYAMGWYRQAPGKEREWVCAISGSGGSTYYADS VKGRFTCSRDNSKNTLYLQMNSLKPEDTAVYYCA (SEQ ID NO: 5); and b) the amino acid sequence of WGQGTQVTVSS (SEQ ID NO: 4) comprising 0, 1, 2, 3, 4 or 5 substitutions. In some embodiments, the VH domain comprises the amino acid sequence of SEQ ID NO: 5 and 4. In some embodiments, the VH domain comprises a) an amino acid sequence having at least 80%, 85%, 90%, 95%, 97%, 97%, 98% 99% or 100% identity to EVQLQESGGGLVQPGGSLRLSCAASGFTFSSYAMGWYRQAPGKEREWVCAISGSGGSTYYADS VKGRFTCSRDNSKNTLYLQMNSLKPEDTAVYYCA (SEQ ID NO: 5); and b) the amino acid sequence of DYWGQGTQVTVSS (SEQ ID NO: 6) comprising 0, 1, 2, 3, 4 or 5 substitutions. In some embodiments, the VH domain comprises the amino acid sequence of SEQ ID NO: 5 and 6. In some embodiments, the CDR3 region comprises 5 to 20 amino acid residues. In some embodiments, the FW3-CDR3-FW4 portion of the VH domain comprises the amino acid sequence of 92-CARX(5 to 15)FDYW-103, wherein X(5 to 15) stands for 5 to 15 amino acid residues. In some embodiments, the CDR3 loops all begin and end with the CAR and FDYW motifs, respectively. Between these conserved motifs, 5 to 15 randomized amino acids (AAs) are inserted, resulting in VH domains with CDR3 loop lengths of 10 to 20 AAs.

In some embodiments, the VH domain comprises the hNb323 scaffold (SEQ ID NO: 8).

In some embodiments, a VH domain described herein comprises a VH3_23 human framework.

In some embodiments of a VH domain described herein, the target of interest is a pathogen derived antigen. In some embodiments, the target of interest is a pathogen derived polypeptide. In some embodiments, the pathogen derived polypeptide comprises a viral, bacterial, or parasitic polypeptide. In some embodiments, the pathogen derived polypeptide comprises a viral polypeptide. In some embodiments, the target of interest is a tumor antigen.

In one aspect, provided herein is a multispecific binding agent comprising at least two VH domains described herein. In some embodiments, the at least two VH domains bind to different epitopes. In some embodiments, the at least two VH domains bind to different epitopes on the same antigen. In some embodiments, the at least two VH domains bind to different antigens. In some embodiments, the multispecific binding agent is a bispecific agent. In some embodiments, the multispecific binding agent is a trispecific agent.

In one aspect, provided herein is a fusion polypeptide comprising at least one VH domain described herein. In some embodiments, the fusion polypeptide of claim 2 comprises more than one VH domain and a heterologous polypeptide. In some embodiments, the heterologous polypeptide is a) an antibody, b) an antibody fragment, c) an Fc domain, d) transmembrane domain, or e) a membrane associating domain. In some embodiments, the heterologous polypeptide is an Fc domain. In some embodiments, the Fc domain is a modified Fc domain. In some embodiments, the Fc domain is a human Fc domain. In some embodiments, the Fc domain comprises a modification that promotes heterodimer formation. In some embodiments, the Fe domain comprises a knob mutation or a hole mutation that promotes heterodimer formation. In some embodiments, the heterologous polypeptide is an antibody. In some embodiments, the heterologous polypeptide is an antibody fragment. In some embodiments the fusion polypeptide is a bispecific tetra-nanobody immunoglobulin (bsNb4-Ig) described herein.

In one aspect, provided herein is a chimeric antigen receptor (CAR) comprising at least one VH domain described herein. In some embodiments, the CAR comprises a target binding domain comprising the VH domain, a transmembrane domain, and an intracellular signaling domain. In some embodiments, the target binding domain comprises a single VH domain. In some embodiments the target biding domain comprises more than one VH domain. In some embodiments, the transmembrane domain comprises a CD8, 41BB or CD28 transmembrane domain. In some embodiments, the intracellular signaling domain is selected from the group consisting of a domain of a human T cell receptor alpha, beta, or zeta chain; a human 41BB domain; a human CD28 domain; and any combination thereof. In some embodiments, the intracellular signaling domain comprises the intracellular domain of a costimulatory molecule selected from the group consisting of CD27, CD28, 41BB, OX40, CD30, CD40, PD1, lymphocyte function-associated antigen-1 (LFA-1), CD2, CD7, LIGHT, NKG2C, NKG2D, B7-H3, a ligand that specifically binds with CD83, and any combination thereof.

In one aspect, provided herein is a host cell comprising a CAR described herein. In some embodiments, the host cell is a T cell or NK cell.

In one aspect, provided herein is a host cell comprising a polynucleotide encoding a CAR described herein. In some embodiments, the host cell is a T cell or NK cell.

In one aspect, provided herein is a polynucleotide encoding the VH domain, multispecific binding agent, fusion polypeptide or CAR described herein. In some embodiments, the polynucleotide is a DNA or RNA. In some embodiments, the polynucleotide is DNA. In some embodiments, the polynucleotide is RNA. In some embodiments, the polynucleotide is an mRNA comprising a modification. In some embodiments, the polynucleotide is an mRNA comprising a modified nucleoside.

In one aspect, provided herein is a vector comprising a polynucleotide described herein. In some embodiments, the polynucleotide encodes a VH domain, multispecific binding agent, fusion polypeptide or CAR described herein. In some embodiments, the vector is a viral vector. In some embodiments, the vector is an adeno associated virus or a lentivirus comprising a polynucleotide encoding a VH domain, multispecific binding agent, fusion polypeptide or CAR described herein. In some embodiments, the vector is a lentivirus comprising a polynucleotide encoding a CAR described herein.

In one aspect, provided herein is a method of producing a VH domain, multispecific binding agent, fusion polypeptide or CAR described herein comprising culturing a host cell comprising a polynucleotide encoding the VH domain, multispecific binding agent, fusion polypeptide or CAR under suitable conditions. In some embodiments, the method produces a VH domain described herein. In some embodiments, the method comprises producing a fusion polypeptide described herein. In some embodiments, the hos cell is a prokaryotic cell, optionally E. coli. In some embodiments, the host cell is a eukaryotic cell, optionally a yeast cell or a mammalian cell, e.g., a T cell or an NK cell.

In one aspect, provided herein is a method of neutralizing a pathogen in a subject in need thereof, comprising administering to the subject a therapeutically effective amount of a composition comprising a VH domain described herein, or a polynucleotide encoding the VH domain.

In one aspect, provided herein is a method of treating a subject in need thereof, comprising administering to the subject a therapeutically effective amount of a composition comprising a VH domain described herein, or a polynucleotide encoding the VH domain.

In one aspect, provided herein is a VH domain library comprising a plurality of VH domains described herein. In some embodiments, the library is a phage library.

In one aspect, provided herein is a polynucleotide library comprising a plurality of polynucleotides encoding VH domains described herein.

In one aspect, provided herein is a method for identifying a VH domain described herein capable of binding a target of interest, comprising a) contacting a VH domain library described herein with a target of interest, and b) identifying a VH domain capable of binding to the target of interest. In some embodiments, the target of interest is a pathogen derived antigen. In some embodiments, the target of interest is a pathogen derived polypeptide. In some embodiments, the pathogen derived polypeptide comprises a viral, bacterial, or parasitic polypeptide. In some embodiments, the pathogen derived polypeptide comprises a viral polypeptide. In some embodiments, the library is a phage library. In some embodiments, the target of interest is a tumor antigen.

In one aspect, provided herein is a method for identifying a polynucleotide encoding a VH domain described herein capable of binding a target of interest, comprising a) expressing a polynucleotide library described herein in a suitable host cell to produce a library of cells expressing a VH domain; b) contacting the library of cells with the target of interest, and c) identifying a polynucleotide encoding a VH domain capable of binding to the target of interest. In some embodiments, the library is a phage library. In some embodiments, the polynucleotide library is expressed in a plurality of yeast cells. In some embodiments, the target of interest is a pathogen derived antigen. In some embodiments, the target of interest is a pathogen derived polypeptide. In some embodiments, the pathogen derived polypeptide comprises a viral, bacterial, or parasitic polypeptide. In some embodiments, the pathogen derived polypeptide comprises a viral polypeptide. In some embodiments, the target of interest is a tumor antigen.

In one aspect, provided herein is a vector comprising a) at least one functional heavy chain V gene, b) at least one heavy chain D gene and at least one heavy chain J gene, and c) a murine C gene which lacks the CH1 exon, wherein the at least one functional heavy chain V gene encodes a polypeptide comprising a fragment of a VH domain described herein, wherein the fragment consists of the residues corresponding to the human VH3_23 gene. In some embodiments, the at least one heavy chain D gene is a human D gene and the at least one heavy chain J gene is a human In some embodiments, the J gene. In some embodiments, the at least one heavy chain D gene is a murine D gene and the at least one heavy chain J gene is a murine J gene. In some embodiments, the at least one functional heavy chain V gene encodes a polypeptide comprising the amino acid sequences of a VH domain described herein. In some embodiments, the amino acid sequences comprise SEQ ID NO: 1, 2, 3, and 4. In some embodiments, the amino acid sequences comprise SEQ ID NO: 5 and 4. In some embodiments, the amino acid sequences comprise SEQ ID NO: 5 and 6. In some embodiments, the vector, further comprising sufficient regulatory sequence elements to enable the at least one functional heavy chain V gene, at least one heavy chain D gene, at least one heavy chain J gene and murine C gene to undergo rearrangement to produce a functional VHH antibody.

In one aspect, provided herein is a transgenic mouse host cell transformed with a vector described herein.

In one aspect, provided herein is a transgenic mouse comprising a vector described herein or a cell comprising a vector described herein. In some embodiments, the mouse comprises one or more of a) a non-functional endogenous lambda light chain locus, b) a non-functional endogenous kappa light chain locus, and c) non-functional endogenous heavy chain locus.

In one aspect, provided herein is a use of the transgenic mouse described herein or the transgenic mouse host cell described herein in producing a VH only antibody or a VH domain that is capable of specifically binding to a target of interest. In some embodiments, the use of the transgenic mouse or the transgenic mouse host cell is in constructing a VH domain library. In some embodiments, the use of the transgenic mouse or the transgenic mouse host cell is in constructing a naïve VH domain library. In some embodiments, the target of interest is a pathogen derived antigen. In some embodiments, the target of interest is a pathogen derived polypeptide. In some embodiments, the target of interest is a tumor antigen. In some embodiments, the pathogen derived polypeptide comprises a viral, bacterial, or parasitic polypeptide. In some embodiments, the pathogen derived polypeptide comprises a viral polypeptide.

Targets of interest: In some embodiments, the target of interest is a pathogen derived antigen. In some embodiments, the target of interest is a pathogen derived polypeptide. In some embodiments, the pathogen derived polypeptide comprises a viral, bacterial, or parasitic polypeptide. In some embodiments, the pathogen derived polypeptide comprises a viral polypeptide. In some embodiments, the viral polypeptide comprises a Betacoronavirus, Chikungunya virus, Dengue virus, Ebola virus, Eastern Equine Encephalitis virus, Herpes Simplex virus, Human Cytomegalovirus, Human Papillomavirus. Human Metapneumovirus, Influenza virus, Japanese Encephalitis virus, Marburg virus, Measles, Parainfluenza virus, Respiratory Syncytial virus, Sindbis virus, Varicella Zoster virus. Venezuelan Equine Encephalitis virus, West Nile virus, Yellow Fever virus, or Zika virus polypeptide or an immunogenic fragment thereof. In some embodiments, the viral polypeptide comprises a MERS-CoV polypeptide, SARS-CoV polypeptide, SARS-CoV-2 polypeptide or an immunogenic fragment thereof. In some embodiments, the viral polypeptide comprises a SARS-CoV polypeptide, SARS-CoV-2 polypeptide or an immunogenic fragment thereof. In some embodiments, the viral polypeptide comprises a SARS-CoV-2 spike protein (S), SARS-CoV-2 envelope protein (E), SARS-CoV-2 nucleocapsid protein (N), SARS-CoV-2 membrane protein (M) or an immunogenic fragment thereof. In some embodiments, the viral polypeptide comprises a SARS-CoV-2 spike protein (S) or an immunogenic fragment thereof. In some embodiments, the target of interest is a tumor antigen.

In one aspect, provided herein are antibodies that specifically bind the SARS-CoV-2 Receptor Binding Domain. In some embodiments, the antibody comprises the VH CDR1, VH CDR2, and VH CDR3 of an antibody described herein. In some embodiments, the antibody comprises the VH domain of an antibody described herein. In some embodiments, the antibody comprises a VH domain having 80%, 85%, 90%, 95%, 97%, 98% or 99% sequence identity to a VH domain described herein, optionally wherein the VH domain comprises VH CDR1, VH CDR2, and VH CDR3 of the VH domain described herein. Also provided herein is a polynucleotide encoding the antibody described herein.

The term “antibody” means an immunoglobulin molecule (or a group of immunoglobulin molecules) that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the terms “antibody” and “antibodies” are terms of art and can be used interchangeably herein and refer to a molecule with an antigen-binding site that specifically binds an antigen.

Antibodies can include, for example, monoclonal antibodies, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), affybodies, Fab fragments, F(ab′)2 fragments, disulfide-linked Fvs (sdFv), anti-idiotypic (anti-Id) antibodies (including, e.g., anti-anti-Id antibodies), bispecific antibodies, and multispecific antibodies. In certain embodiments, antibodies described herein refer to polyclonal antibody populations. Antibodies can be of any type (e.g., IgG, IgE, IgM, IgD, IgA, or IgY), any class (e.g., IgG1, IgG2, IgG3, IgG4, IgA1, or IgA2), or any subclasses (isotypes) thereof (e.g. IgG1, IgG2, IgG3, IgG4, IgA1 and IgA2), of immunoglobulin molecule, based on the identity of their heavy-chain constant domains referred to as alpha, delta, epsilon, gamma, and mu, respectively. The different classes of immunoglobulins have different and well known subunit structures and three-dimensional configurations.

As used herein, the terms “antigen-binding domain,” “antigen-binding region,” “antigen-binding site,” and similar terms refer to the portion of antibody molecules which comprises the amino acid residues that confer on the antibody molecule its specificity for the antigen (e.g., 3FTx-L). The antigen-binding region can be derived from any animal species, such as mouse and humans.

As used herein, the terms “variable region” or “variable domain” are used interchangeably and are common in the art. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises human CDRs and human framework regions (FRs). In certain embodiments, the variable region comprises human CDRs and primate (e.g., non-human primate) framework regions (FRs).

There are several approaches for determining CDRs. One approach is based on cross-species sequence variability (i.e., Kabat et al., Sequences of Proteins of Immunological Interest, (5th ed., 1991, National Institutes of Health, Bethesda Md.), “Kabat”). A second approach was developed by the MGT, the international ImMunoGeneTics database (imgt.cines.fr) as a high quality integrated information system specializing in immunoglobulins (IG), T cell receptors (TR) and major histocompatibility complex (MHC) of human and other vertebrates. Lefranc, M.-P., The Immunologist, 7, 132-136 (1999). The IMGT unique numbering defined for the IG and TR variable regions and domains of all jawed vertebrates has allowed a redefinition of the limits of the framework (FR-IMGT) and complementarity determining regions (CDR-IMGT), leading to a standardized description of mutations, allelic polymorphisms, 2D representations (Colliers de Perles) and 3D structures, whatever the antigen receptor, the chain type, or the species. A third approach is based on crystallographic studies of antigen-antibody complexes (Al-lazikani et al, J. Molec. Biol. 273:927-948 (1997)). In addition, combinations of these approaches are sometimes used in the art to determine CDRs. In some embodiments, the CDR regions are determined according to Kabat. In some embodiments, the CDR regions are determined according to IMGT.

The Kabat numbering system is generally used when referring to a residue in the variable domain (approximately residues 1-107 of the light chain and residues 1-113 of the heavy chain) (e.g., Kabat et al., Sequences of Immunological Interest. (5th Ed., 1991, National Institutes of Health, Bethesda, Md.) (“Kabat”).

The term “antibody fragment” refers to a portion of an intact antibody. An “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies.

### EXAMPLES

Described herein is a human-based Nb library, which was validated using SARS-CoV-2 as a test antigen. Also described is an on-yeast competitive selection strategy paired with deep sequencing to rapidly determine the specificity of the selected Nbs. The epitope binning information was then used to select candidate Nbs targeting non-overlapping epitopes that can be incorporated into a chimeric Nb/Ab hybrid architecture by replacing the conventional Ab V_{H} and V_{L} domains with different Nbs. This modular format facilitates bispecific Nb creation through the native Ig C_{H}1/C_{L} domain pairing and allows for the rapid production of different Nb combinations by mixing different plasmids during the transfection. The resulting bispecific tetra-nanobody immunoglobulin (bsNb4-Ig) can achieve multivalent interactions through the two Nbs on a single “Fab” fragment and/or through the contribution of Nbs on adjacent Fabs arms of the molecule. Furthermore, Nbs that recognize non-overlapping epitopes can facilitate biparatopic engagement of a single target molecule, provided the linkers that connect the Nbs to the constant domain are sufficiently long. Presence of the Fc domain also enables the use of conventional purification strategies during production and provides the benefits of Fc interactions in vivo. Also disclosed herein the structural characterization of selected Nbs to confirm the mode of interaction compared to conventional Abs. Additional information regarding the characterization of the anti-SARS-CoV-2 antibodies described herein can be found in Mindrebo et al., PNAS 120(24): e2216612120 (2023) and supporting information, each of which is incorporated herein by reference for all purposes.

### hNb323 Construction and Validation

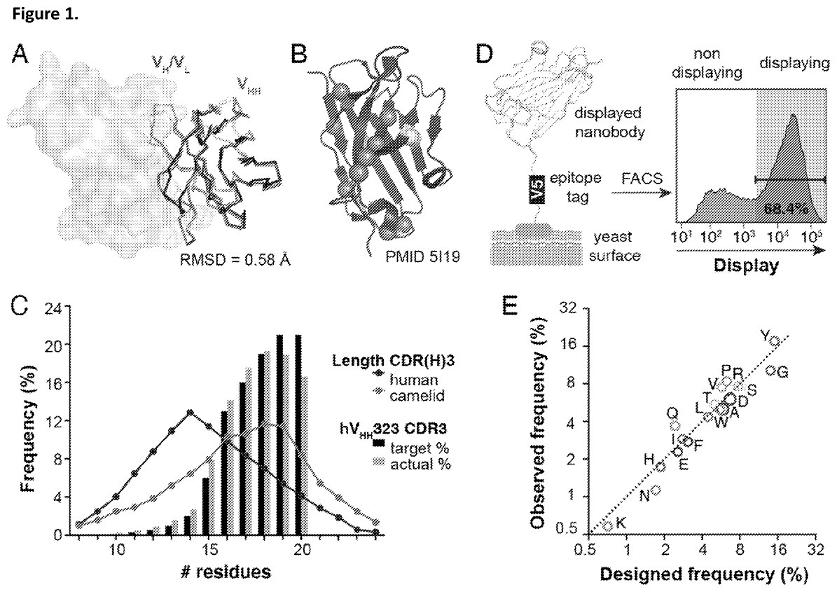

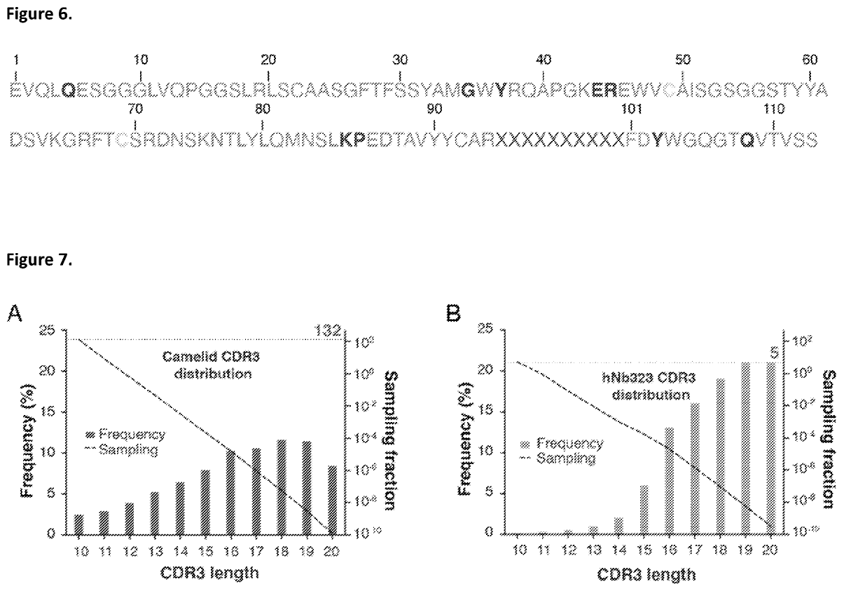

The human-based Nb library was designed to have a conserved variable domain (including CDR1 and CDR2) with all the diversity localized to the CDR3 loop, mimicking the diversification of a naive immune repertoire. Based on the high sequence identity between camelids V_{HH}s and human V_{H}3s (15) (FIG. 1A), a synthetic V_{HH} scaffold was developed by incorporating common V_{HH} mutations into the human V_{H}3-23 heavy chain (HC) gene (FIG. 1B). The starting scaffold (hV_{H}323) was modified by including previously reported “camelizing” mutations that remove the need for light chain (LC) pairing (16-19), and mutations from consensus V_{HH} sequences that removed surface exposed hydrophobic residues (FIG. 6). To further stabilize the V_{HH} scaffold, an additional internal disulfide bond was introduced by mutating Ser49 and Ile69 to Cys (20).

The CDR3 loops all begin and end with the CAR and FDYW motifs, respectively. Between these conserved motifs, 5 to 15 randomized amino acids (AAs) are inserted, resulting in Nbs with CDR3 loop lengths of 10 to 20 AAs. The AA frequencies in the CDR3 loops were designed based on the non-templated AA frequencies found in naturally occurring Abs and Nbs, excluding Met and Cys to avoid producing Nbs with unwanted chemical liabilities. CDR3 lengths were distributed following a sigmoidal distribution rather than mimicking the naturally occurring normal distribution (FIG. 1C). The right-shifted distribution was selected because the theoretical diversity of the shorter CDR3 loops is relatively small and would be oversampled if a normal distribution were used (FIG. 7).

DNA encoding the human-based Nb library was transformed into a yeast surface display vector via homologous recombination with an estimated diversity of 3×10⁹ based on a colony formation assay performed after the transformation. The Nbs displayed well on the yeast surface, with ~67% of the library showing surface display and a median 180-fold increase in signal between the displaying cells and nondisplaying cell populations (FIG. 1D). Deep sequence analysis of Nb encoding DNA recovered from the transformed cells showed a good agreement with the target library specifications. Both the targeted CDR3 length and AA distributions agreed well with the observed distributions (FIG. 1C, E).

### Discovery of Binders Using SARS-CoV-2 Receptor Binding Domain (RBD)

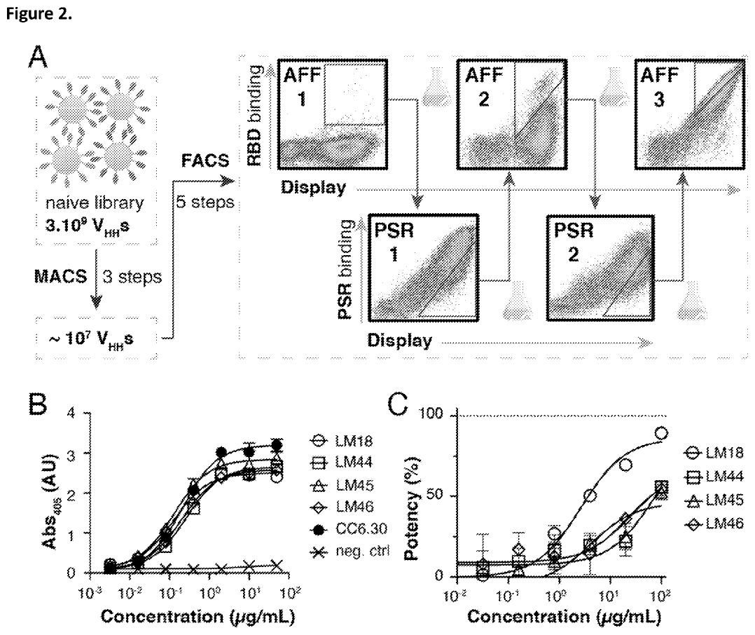

Upon completion of the library, it was validated using SARS-CoV-2 receptor binding domain (RBD) as a test antigen. The overall library screening strategy was performed using a combination of Magnetic-Activated Cell Sorting (MACS) followed by Fluorescence-Activated Cell Sorting (FACS) (FIG. 2A). After each round of selections, the collected cell population was expanded and re-induced prior to the next round. The naive library was too large to be screened using conventional FACS, so three rounds of MACS were used to bulk enrich the small number of SARS-CoV-2 reactive clones. RBD-reactive cells were enriched in the first round of MACS by labeling 1×10¹¹ cells from the induced library (a 30-fold oversampling of the theoretical diversity) with biotinylated SARS-CoV-2 RBD. Excess antigen was removed, and the cells were then incubated with streptavidin-conjugated magnetic microbeads. Cells bound to the magnetic microbeads were magnetically enriched, expanded, and re-induced for additional rounds of selection. In this first positive MACS selection, the collected cells were enriched for SARS-CoV-2 RBD reactive clones, but also streptavidin and microbead reactive clones. To deplete the latter two populations, the second round of MACS also included a counter selection where induced cells were incubated with the streptavidin-conjugated magnetic microbeads only and the nonreactive population was collected. The microbead-depleted cells were then enriched for RBD-reactive clones using another positive MACS selection.

After enrichment for RBD binding Nbs by MACS, five additional rounds of multicolor FACS were used to isolate clones with high binding affinity and specificity for RBD. In FACS rounds 1, 3 and 5 (AFF1, 2 and 3), cells were labeled with sub-saturating concentrations of biotinylated RBD followed by fluorescently conjugated streptavidin and an anti-V5 Ab to quantify the amount of RBD and amount of surface displayed Nb, respectively. Normalizing for the level of Nb displayed on the surface of the cell, the high RBD binding clones were enriched. To help ensure the selected Nbs were specific for RBD, negative selections were interspersed at FACS round 2 and 4 (PSR1 and 2) to remove polyreactive “sticky” clones. In this counterselection, cells were labeled with a complex preparation of biotinylated and detergent solubilized HEK cell membrane proteins (polyspecificity reagent or PSR) and the PSR-low fraction was collected, again normalizing for the level of Nb display on the cell surface (FIG. 2A).

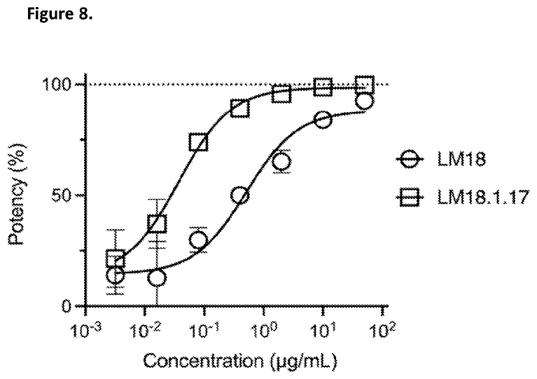

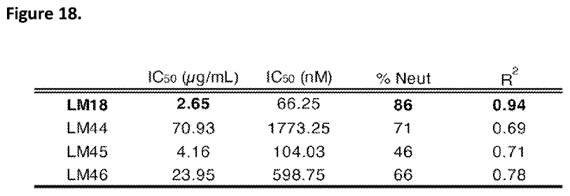

As this was the first antigen screened in this library, it was used to test the specificity and biochemical behavior of the selected Nbs before conducting a more in-depth analysis of the enriched population. Sanger sequencing of 96 clones found the library to be highly enriched, recovering a total of four unique clones. These Nbs were expressed in HEK cells as just the Nb and as a fusion protein to a human Fc. Nbs expressed well in both formats, producing a normalized expression titer of 50-200 mg/L and 220-450 mg/L for the Nb and Fc fusions, respectively. Both the Nbs and Nb-Fc fusions were monodispersed by analytical SEC. Enzyme-linked immunosorbent assay (ELISA) confirmed RBD specificity was confirmed for the four variants (FIG. 2B), and no polyspecific binding was observed in the standard assays. Finally, the four Nb-Fc fusions were tested in a SARS-CoV-2 pseudovirus (PSV) neutralization assay and found that one of the Nbs, LM18, neutralized with an IC₅₀ of 2.65 μg/mL (66 nM) (FIG. 2C, FIG. 18). Interestingly, LM18 also neutralized SARS-CoV-1 PSV with an IC₅₀ of 0.5 μg/ml (or 12 nM) (FIG. 8) as it serendipitously targeted a conserved epitope between the two variants.

### FACS-Based Epitope Binning and Deep Sequencing to Determine Library Specificities

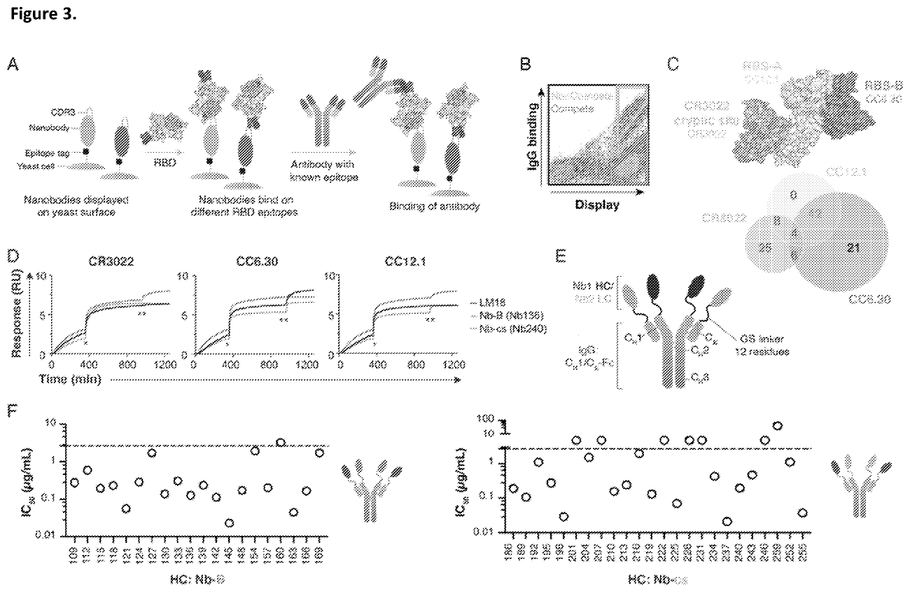

After confirming that the base hVr323 could produce biochemically well behaved Nbs that bound a target antigen with high specificity, it was determined how many Nbs were enriched during the selections and what epitopes on RBD were targeted by these Nbs. Epitope specificity was determined using an on-yeast competition assay with three structurally characterized Abs that target the RBS-A (CC12.1), RBS-B (CC6-30), and CR3022 epitopes on SARS-CoV-2 RBD (FIG. 3A-C) (23, 24). Cells from the outgrowth following the FACS 4 negative selection (PSR2) were analyzed, as all clones in this population were RBD-reactive and had undergone both polyreactive negative selections, thus, should have relatively few sticky clones. The induced library was labeled with SARS-CoV-2 RBD, then incubated with one of the three anti-SARS-CoV-2 Abs and followed by fluorescently conjugated anti-human IgG fluorescently conjugated secondary Ab. In this format, Nbs that recognized overlapping epitopes with the human IgGs blocked binding and both the competitive (C) and non-competitive (NC) populations were collected. The starting population and all 6 C and NC selections were deep sequenced and after filtering to remove likely sequencing artifacts, 123 unique Nbs were observed in the dataset. Epitope bins were assigned by defining an overlapping epitope with a tested SARS-CoV-2 Ab as having a C/NC ratio>10, and C/NC ratio<10 for a nonoverlapping epitope. From the 123 unique CDR3s identified, 81.3% have a clear competitive binding profile with at least one of the three tested Ab (FIG. 3C), whereas 10.6% bind to an epitope outside of RBS-A and -B and CR3022 cryptic site, as they were NC with all three Abs. Four and six Nbs were identified to map to CR3022/CC12.1/CC6.30 and CR3022/CC6.30 compete, respectively, and these Ab epitopes should not simultaneously compete based on structural analysis. While it cannot be excluded that binding of those Nbs might induce a conformational change in RBD and therefore prevent the binding of the competitive Abs, the epitopes targeted by these Nbs remain ambiguous.

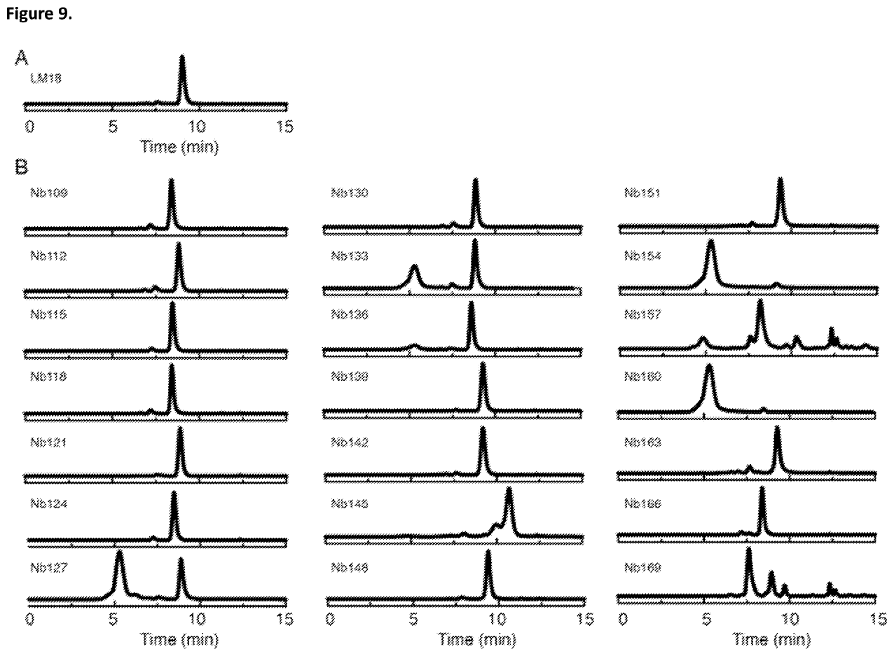

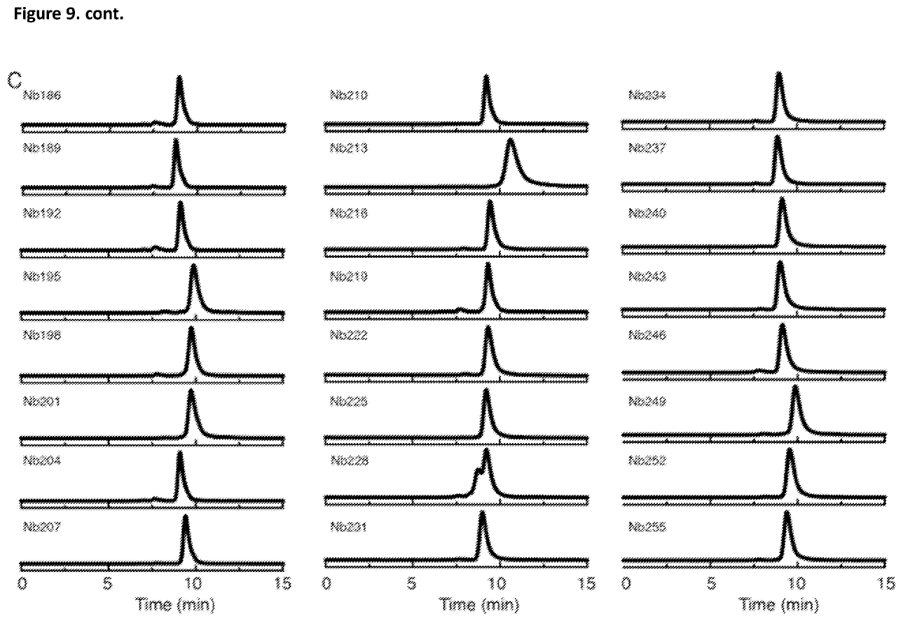

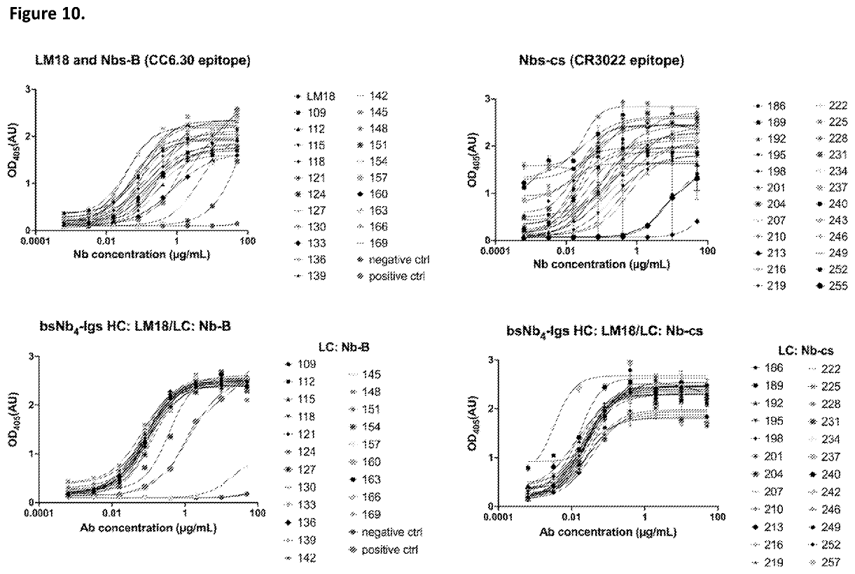

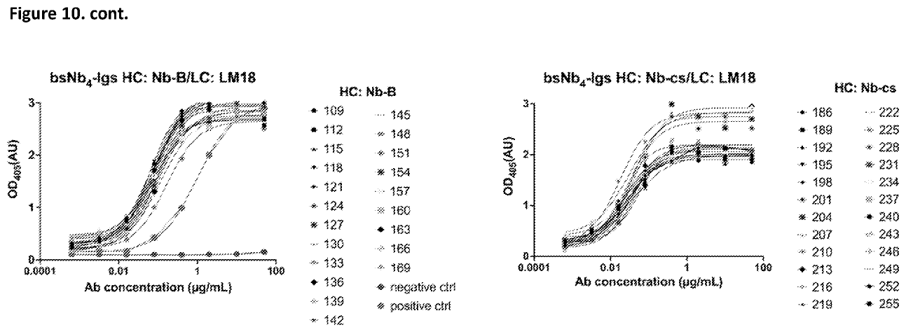

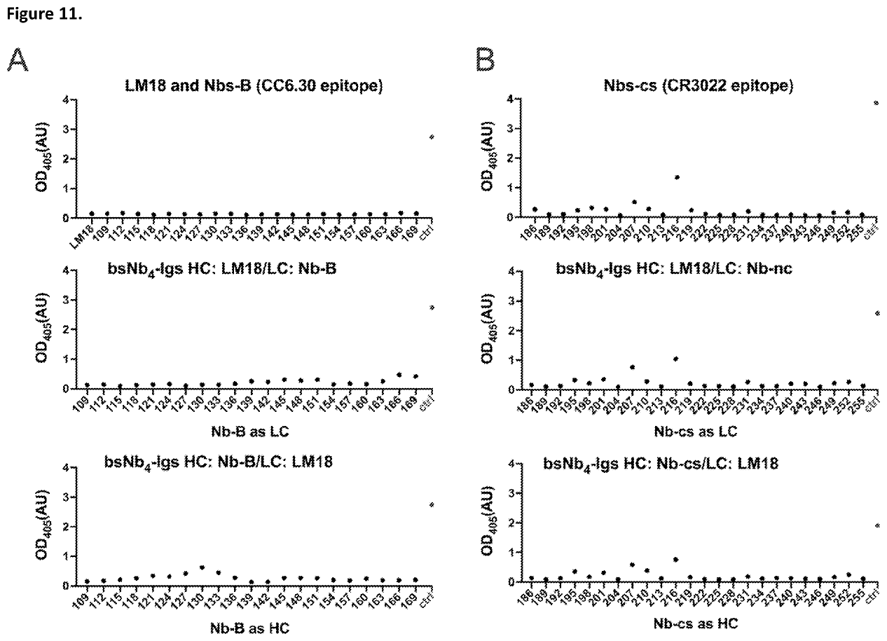

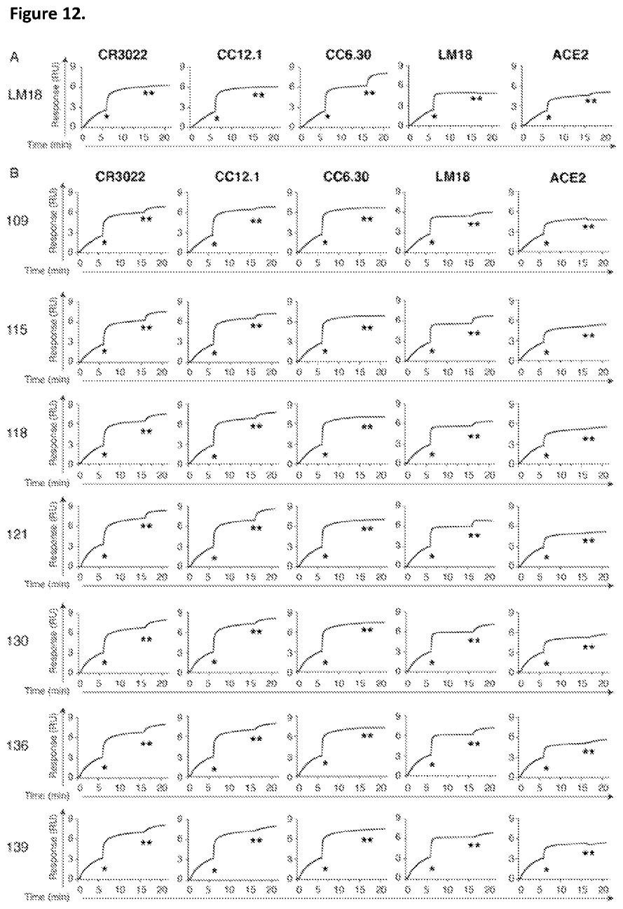

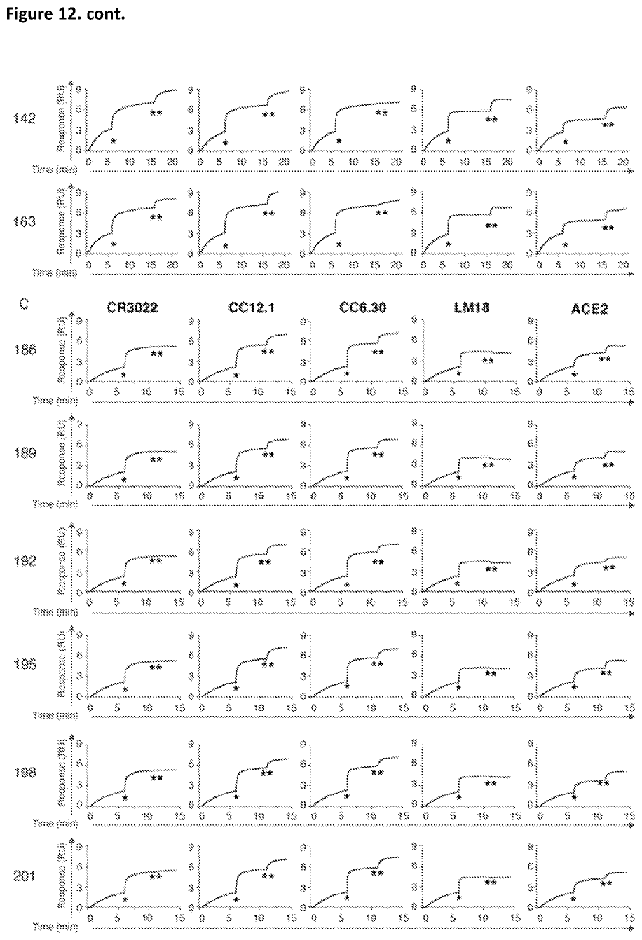

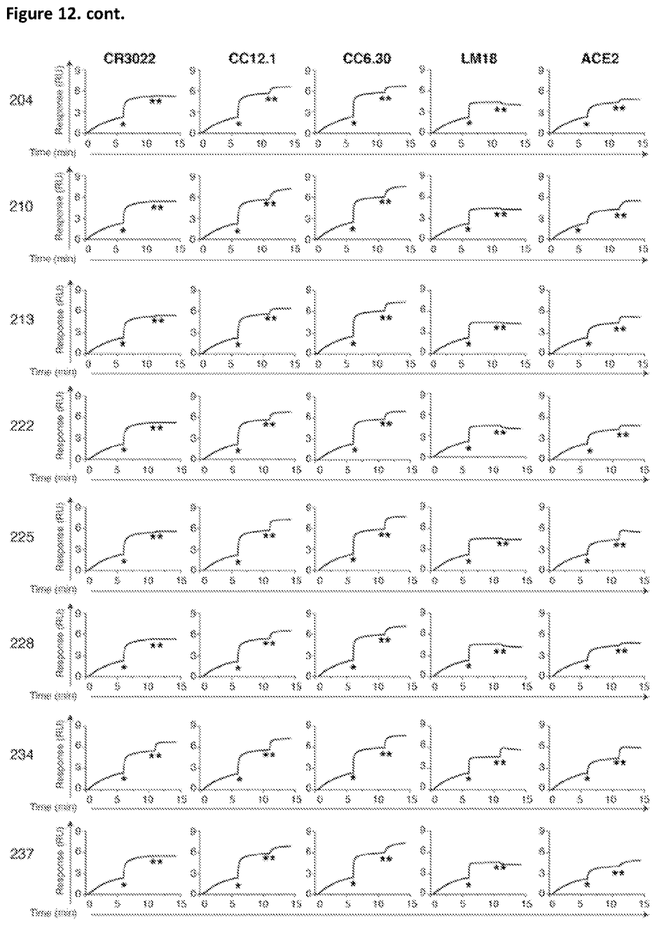

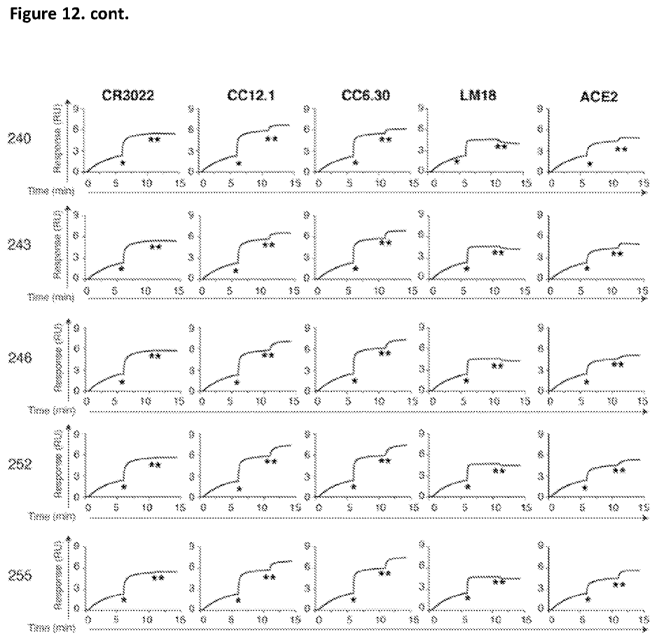

A total of 45 Nbs, 21 Nbs targeting the RBS-B epitope and 24 Nbs targeting the CR3022 epitope, were selected for expression and validation as the same Fc fusions described above. The primary intent was to determine whether the on-yeast epitope binning correlated well with conventional binning, but also to get an extended sampling of the biochemical behavior of Nbs coming from this library. Nbs were expressed as Fc fusion constructs, their size distribution was checked by size exclusion chromatography (SEC) (FIG. 9) and specific binding to SARS-CoV-2 RBD was confirmed by ELISA (FIGS. 10 and 11). To evaluate the accuracy of the FACS-based epitope binning, biolayer interferometry (BLI)-based assays were performed with the three Abs used for the selection (CR3022, CC12.1 or CC6.30). The results are consistent with the yeast cell binning and validate the approach used to select the Nbs (FIGS. 3D, 12).

### Using Epitope Information to Develop Bispecific Nbs

To improve the functional properties of the selected Nbs strategies were sought to format several Nbs into a single molecule, aided by the above epitope information. It has been shown that Nbs can be readily assembled into multimers using peptide linkers which can result in improved binding, and consequently, neutralization (6, 9, 10, 13, 14). Multimerizing a single Nb can improve neutralization by facilitating inter-spike and/or intra-spike crosslinking (9), and incorporating Nbs targeting non-overlapping epitopes has the potential added benefit of facilitating biparatopic interactions with a single subunit and may be more resistant to antigenic diversity (6, 25). Our approach to combine multiple Nbs was to construct a bispecific tetra-nanobody-based Igs (bsNb₄-Ig) by replacing the Ab V_{H} and V_{L} domains with two Nbs that target distinct RBD epitopes (FIG. 3E) (26-30). Nbs were linked to the C_{H}1 and C_{K} domains by a flexible glycine/serine linker, ensuring a 2:2 ratio that could be easily expressed and purified using standard antibody production protocols. This linkage potentially facilitated simultaneous Nb engagement on a single RBD, on multiple RBDs with SARS-CoV-2 spike trimer or potentially through inter-spike crosslinking (10, 28).

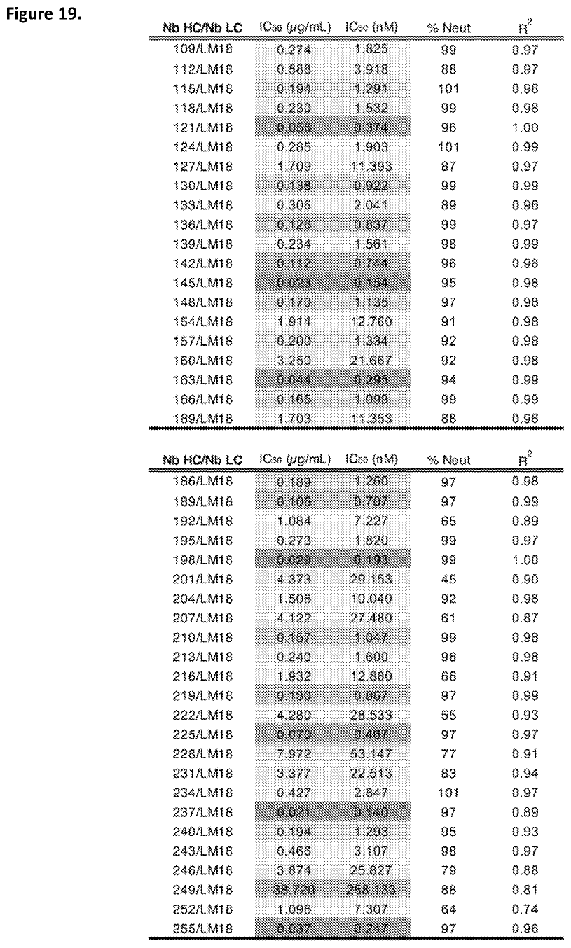

Based on the preliminary analysis of LM18 and its interesting breadth of neutralization for both SARS-CoV-1 and SARS-CoV-2 PSVs, bispecific designs around LM18 were constructed. Deep sequencing analysis found that LM18 competed with CR3022 and CC12.1 but not CC6.30, suggesting that it targeted an epitope similar to ADG20 (31). This specificity indicated that Nbs targeting the RBS-B epitope (competes with CC6.30 only) should be capable of biparatopic RBD engagement when paired with LM18. It was also hypothesized that some Nbs that targeted the CR3022 cryptic site (CR3022 compete only) may also be able to simultaneously engage with LM18, as LM18 likely targets the edge of the CR3022 epitope. All the Nbs showing both specific RBD binding and monodisperse size-exclusion profiles by SEC were reformatted into the bsNb₄-Ig format with a Nb-B or a Nb-cs as the HC, and LM18 as the LC. SARS-CoV-2 RBD binding was first confirmed by ELISA (FIGS. 10 and 11) and then tested in a SARS-CoV-2 PSV neutralization assay. 16 out of the 20 Nb-B/LM18 bsNb₄Ig and 13 out of the 23 Nb-cs/LM18 bsNb₄-Ig showed at least a 10-fold (1 μg/mL, 7 nM), and up to a 470-fold (14 ng/ml, 0.14 nM) improvement in neutralization IC₅₀ compared to LM18 (FIG. 3F, FIG. 19).

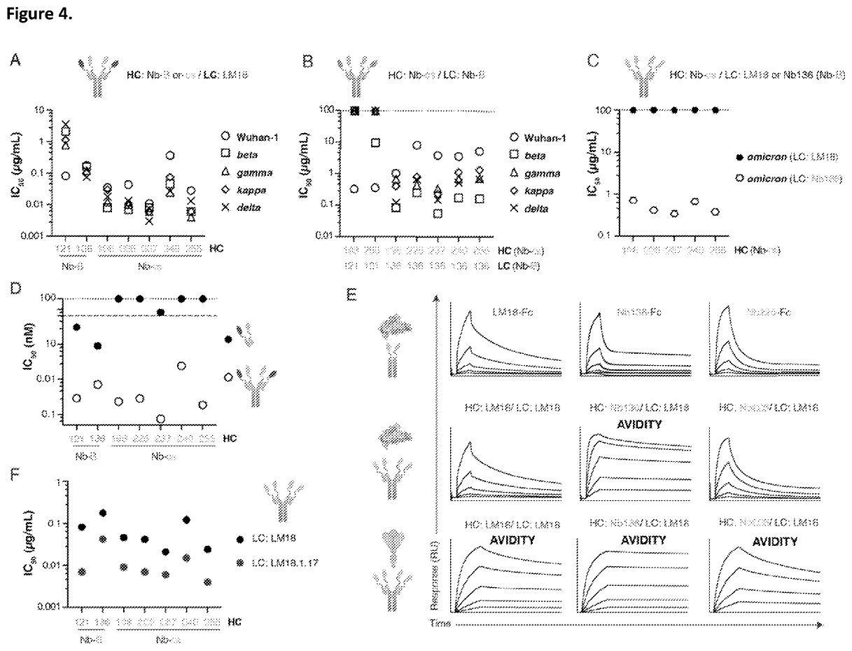

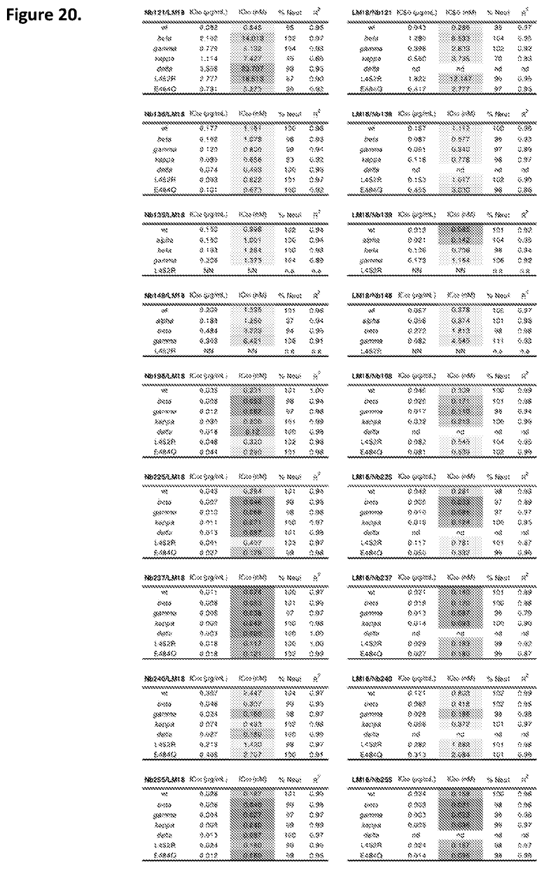

Neutralization breadth of 10 bsNb₄Igs was next measured across a panel of SARS-CoV-2 PSV variants (FIG. 20). Initially, all 10 bsNb₄-Igs were screened against PSV variants containing the single L452R (present in kappa and delta variants) and E484Q (present in kappa variant) point mutations, with two out of the five Nb-B/LM18 bsNb₄-Igs and all five Nb-cs/LM18 bsNb₄-Igs neutralized these variants. This neutralization profile was consistent with the epitope binning data, as the tested mutations were located in the RBS-B epitope, but also demonstrated that the Nbs within an epitope have subtly different specificities that can enhance or reduce their susceptibility to antigenic variability. The seven bsNb₄-Igs that neutralized the L452R and E484Q mutants were then tested against the full PSVs for SARS-CoV-2 beta (B.1.351), gamma (P.1), kappa (B.1.617.1), and delta (B.1.617.2) variants (FIG. 4A). Although the neutralization potency varied across the bsNb₄Igs in the panel, 6/7 in the panel neutralized all 5 PSVs with an IC₅₀<1 μg/mL. Several of the bsNb₄-Ig where LM18 was paired with an RBD-cs targeting Nb (LM18 as HC and Nb198, Nb225, Nb237 or Nb255 as LC) neutralized all the PSVs with an average IC₅₀ of 30 ng/ml (0.2 nM).

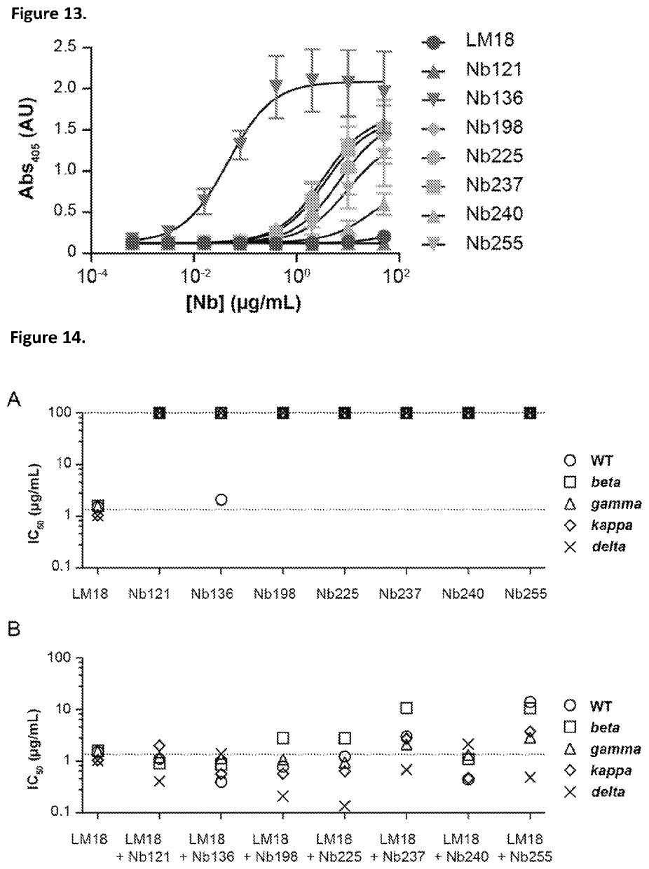

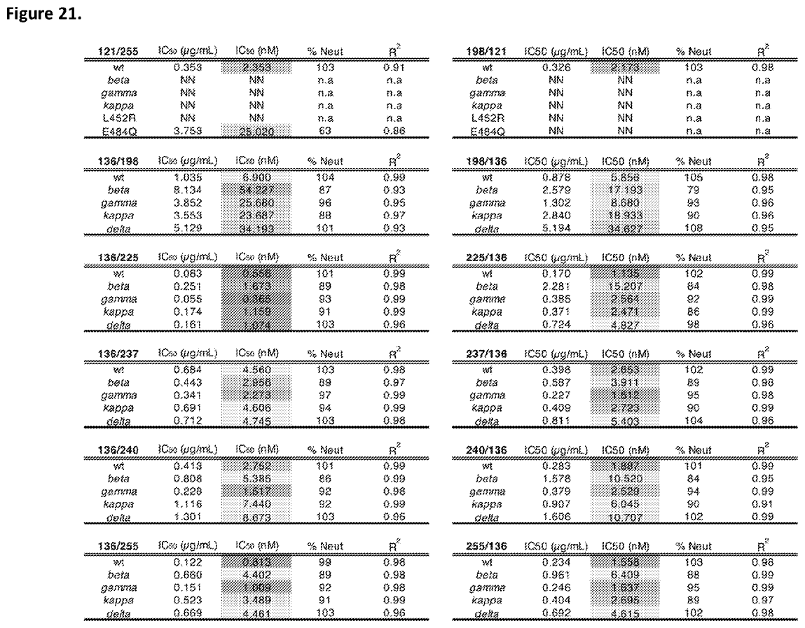

Seven bsNb₄-Igs were next constructed from the Nb-B and Nb-cs building blocks used in the LM18-based bispecifics. RBS-B epitope targeting Nb121 or Nb136 were combined with RBD-cs targeting Nbs Nb198, Nb225, Nb237, Nb240 and Nb255. All five of Nb136-based bsNb₄-Igs could neutralize the five viral variants, albeit slightly less potently than LM18 combined with RBD-cs targeting Nbs (FIG. 4B, FIG. 21). When the omicron variant was reported, all the bsNb₄-Igs reported here were tested and were found that bsNb₄-Ig with Nb136 on one arm and an RBD-cs targeting Nb on the other could neutralize this new variant. All the bispecifics that included LM18 were unable to neutralize due to omicron being resistant to LM18 (FIGS. 4C, 13).

### Neutralization Potency Gains From Tetravalent-Ig Format

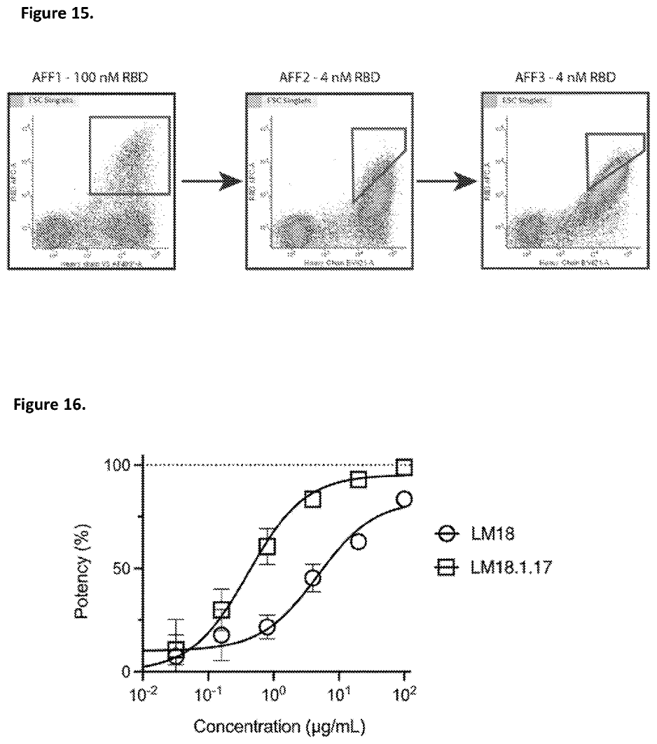

Several of the bsNb₄-Igs constructs were able to achieve neutralization breadth and potency comparable to best-in-class Abs recovered from SARS-CoV-2 convalescent donors (32, 33) and tests were designed to better understand what enabled this function. First, all eight of the Nb building blocks were tested in the bsNb₄-Igs expressed as homomeric Fc fusions for the ability to neutralize Wuhan-1 SARS-CoV-2 PSV. Both LM18-Fc and Nb136-Fc neutralized while the other 6 Nbs-Fc did not at the maximum concentration of 100 μg/mL (2.5 μM), ruling out the possibility that the tetravalent bsNb₄Igs potency was due to one of the Nbs with potent neutralizing ability (FIG. 14). Next, cocktails of homodimeric Fc fusions with equimolar equivalents of LM18 and either Nb-B or Nb-cs Nbs were tested (FIG. 15). The neutralization potency of these cocktails was roughly equivalent to the LM18-Fc fusion alone, indicating that the increased potency was not due to synergy between the two Nb specificities in the bsNb₄-Igs but that it required them to be on a single molecule.

To test the contributions of the two Nb specificities formatted into a single molecule, molecular “Fabs” were produced consisting of a single arm from the bsNb₄-Igs. Bispecific Nb-Fabs with LM18 fused to the C_{H}1 domain and one of the RBS-B targeting Nbs fused to the C_{L} domain showed modest increase in neutralization potency compared to the homomeric Fc fusions with LM18, the Nb-B or a cocktail of the two Fc fusions (FIG. 4D). In contrast, four of the five bispecific Fabs with a Nb-cs fused to the C_{L} domain failed to neutralize at concentrations of 100 μg/mL (1.9 μM), and the sole variant that showed any neutralization (Fab LM18/Nb237) had reduced potency compared to the LM18 homomeric Fc fusion. These findings were unexpected, as the LM18/Nb-cs bsNb₄-Igs were more potent than the LM18/Nb-B bsNb₄-Igs (FIG. 3A) despite the fact that Nb-cs and LM18 cannot engage the same RBD simultaneously (FIG. 12).

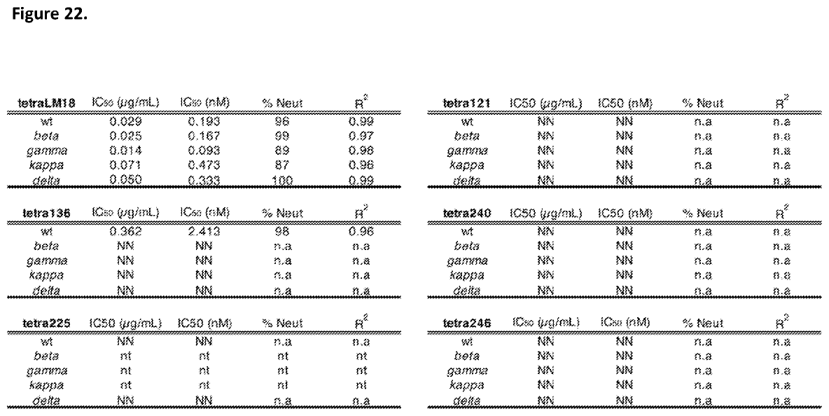

Lastly, the tetravalent Igs with four copies of the same Nb (Nb₄Igs) were expressed. In this format, LM18-Nb₄Ig showed a 340-fold increase in potency compared to the LM18 bivalent homomeric Fc fusion against Wuhan-1 SARS-CoV-2 PSV (IC₅₀ of 0.029 μg/mL or 0.2 nM). 136-Nb₄Ig also showed a 20-fold increase in neutralization potency over the corresponding bivalent homomeric Fc fusion (2.1 μg/mL or 53 nM) with and IC₅₀ of 0.36 μg/mL (2.4 nM) for SARS-CoV-2 PSV. 121-Nb₄Ig, 225-Nb₄Ig, 240-Nb₄Ig and 246-Nb₄Ig did not neutralize at concentrations of 100 μg/mL (FIG. 22). These results highlight the LM18 specific epitope and neutralization properties, but also the advantages of bispecific constructs rather than monospecific tetravalent for neutralization, as several of the LM18/NbC bsNb₄Ig neutralized more potently than LM18-Nb₄Igs across the panel of SARS-CoV-2 PSVs.

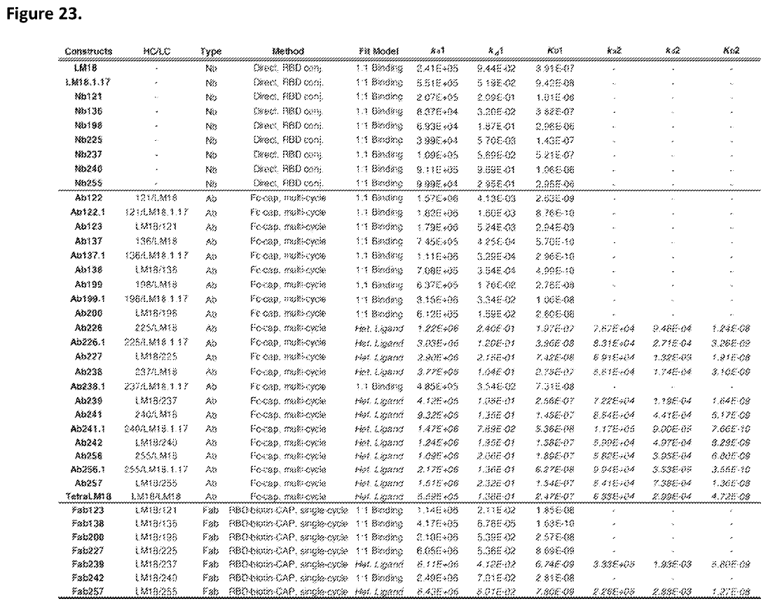

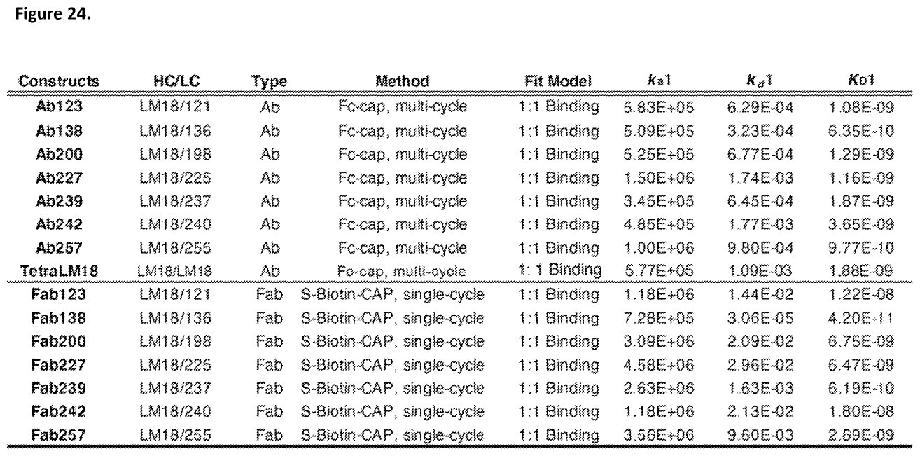

To evaluate the correlation between binding affinity and neutralization potency, the binding affinities of the Nb building blocks LM18, Nb136 and Nb225 as well as the corresponding bsNb₄-Igs for SARS-CoV-2 RBD were determined by surface plasmon resonance (SPR). All three of the individual Nbs had relatively weak binding for SARS-CoV-2 RBD, with K_{D}s between 143 nM and 391 nM and exhibiting a rapid dissociation rate, binding kinetics commonly observed with synthetic Nbs (FIGS. 23 and 24). In the tetra-Ig format, LM18-Nb₄Ig and the LM18/Nb225 bsNb₄-Ig had kinetics that were similar to the individual Nbs (FIG. 4E). The LM18/Nb136 bsNb₄-Ig showed a 630-fold improvement in K_{D} to 500 pM due to a nearly non-existent off-rate, consistent with the epitope binning that showed LM18 and Nb136 bind to non-overlapping epitopes and could be capable of biparatopic engagement (FIG. 3D). Finally, the prefusion-state stabilized SARS-CoV-2 S trimer (33) was tested against all three Nb-Ig formats, all of which bound with high affinity and had a very slow dissociation rate. Of note, the LM18/Nb136 had a notably slower dissociation rate compared to the other two variants, yet this bsNb₄-Ig was the least potent neutralizer of the group. The mode of binding for the S trimer to the bsNb₄-Ig is unclear and likely heterogeneous, however, it is clear that all tetra-Igs are able to achieve intra-spike avidity and substantially reduce the apparent off-rate. There was nothing in the binding data to suggest why LM18/Nb-cs variants would neutralize as potently as they do, but this may be partially attributed to the RBD subunits being preferentially in the “down” conformation on the stabilized S trimers (34).

### Affinity Maturation of Nb Building Blocks

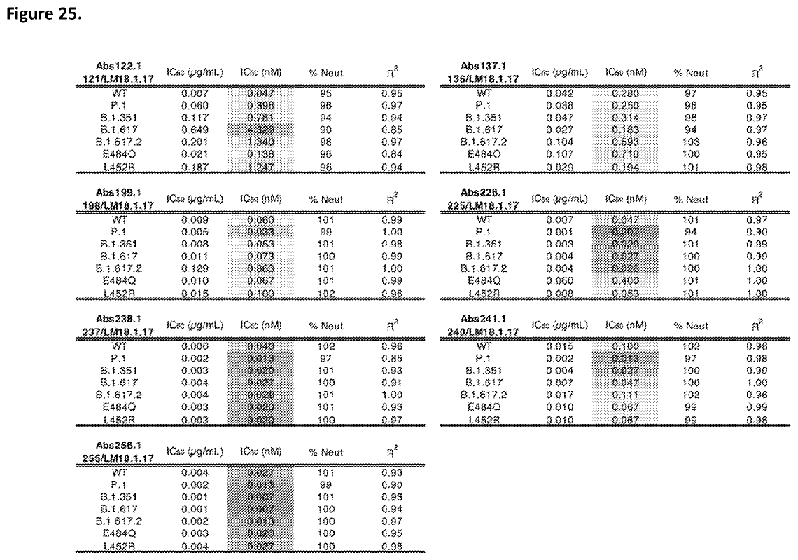

All the above work explored how the Nb potency can be improved through modular multivalent formats, however, it is well established that improving Nb affinity for a target antigen also can improve potency. It was hypothesized that affinity maturation of the base Nbs, specifically improving the dissociation rate, would translate to improvements in potency for the resulting bsNb₄-Igs. To test this, an affinity maturation library of LM18 was created using the SAMPLER maturation strategy (35), where one all single mutation variants of each CDR loop are generated and combined into a combinatorial library. This library was displayed on the surface of yeast and subjected to four rounds of FACS selections—two rounds with RBD to enrich higher affinity clones, one PSR round to deplete sticky clones, and a final round with RBD to further enrich higher affinity clones (FIG. 15). At the conclusion of the selections, 96 colonies were submitted for Sanger sequencing. No clear consensus sequence was present, therefore 10 constructs were selected for recombinant production as Fc fusions. The IC₅₀ value of the best variant, LM18.1.17, improved by a factor of 6 (0.41 μg/mL or 10 nM), and 13 (0.037 μg/mL or 0.9 nM) for the neutralization of SARS-CoV-2 and SARS-CoV, respectively (FIGS. 8, 16). SPR analysis of LM18.1.17 binding to RBD also identified a 6-fold improvement in binding affinity compared to the parental LM18 (FIG. 23). LM18.1.17 instead of LM18 was then incorporated into the bsNb₄-Igs format and measured the neutralization potency against SARS-CoV-2 PSV (FIG. 4F, FIG. 25). In all cases, LM18.1.17-based bsNb₄-Igs showed more potent neutralization compared to LM18-based bsNb₄-Igs. As expected, affinity maturing the base Nb components translated to potency improvements of the bsNb₄-Igs, demonstrating that the overall function can be further improved.

### Structural Validation of Engineered Nb Design

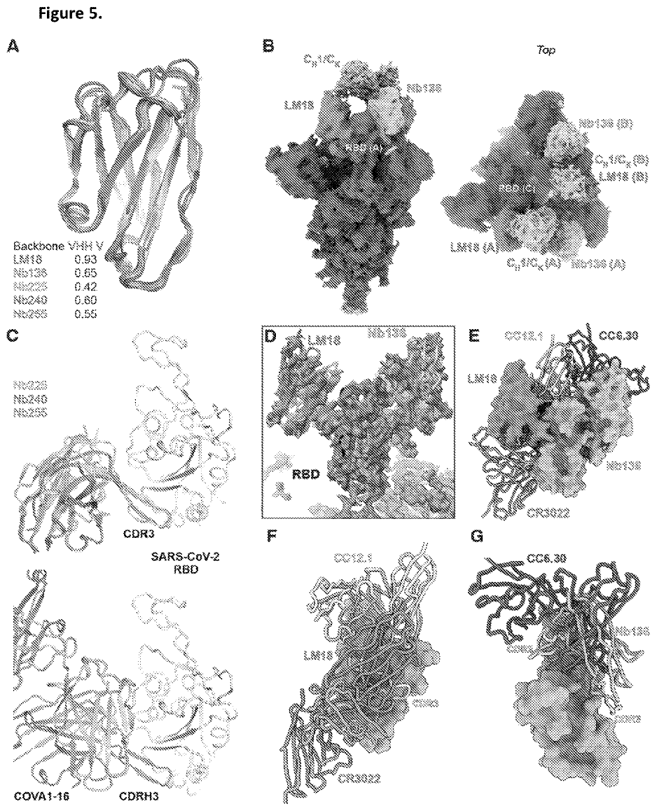

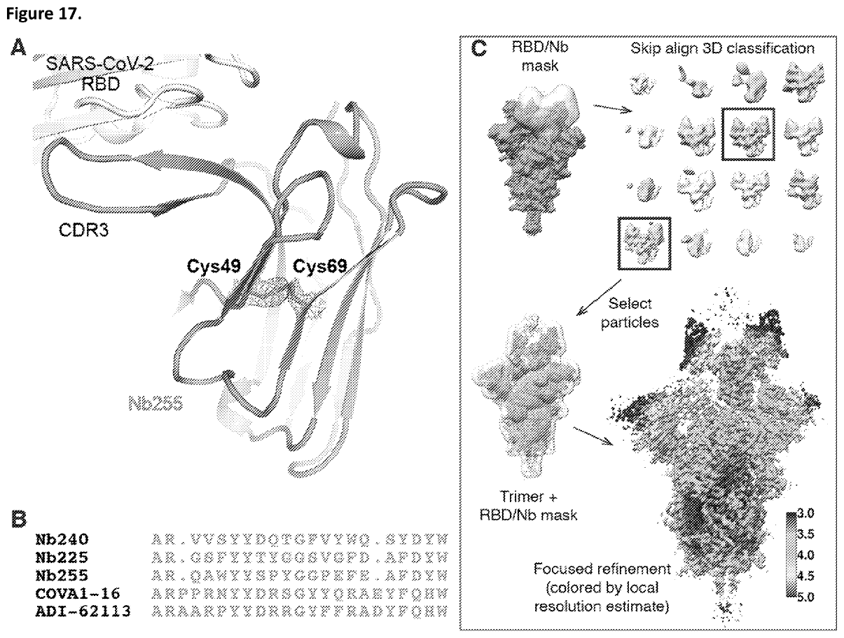

To understand the structural integrity and epitope propensity of the engineered Nbs, atomic structures of the lead Nbs in complex with SARS-CoV-2 RBD (x-ray crystallography) and SARS-CoV-2 S protein (cryo-electron microscopy) were determined. The structures reveal that these Nbs fold in a V_{HH} fold as expected (FIG. 5A). The mean root-mean-square-deviation (RMSD) of protein backbone structures between the five engineered Nbs and a representative camelid Nb (e.g. V_{HH} V) is 0.63 (excluding CDR3), suggesting that the synthetic designs mimic naturally occurring Nbs in terms of overall topology. The engineered Cys49 and Cys69 disulfide designed to maintain the structural stability in these Nbs is evident in the x-ray crystallographic electron density maps (FIG. 17).

For bsNb₄-Ig bispecific engineering constructs, a cryo-EM structure of LM18/Nb136 in complex with trimeric S protein demonstrates that a single Ig arm (containing one copy each of LM18 and Nb136) can simultaneously engage two binding sites on the same up RBD, and a single bsNb₄-Ig appears to bind 4 sites total across two up RBDs from the same trimer (FIG. 5B). Such efficient engagement of the tetravalent LM18/Nb136 is consistent with the slower dissociation rate compared to other bsNb₄-Ig combinations, although the requirement of at least 2 RBDs in the up conformation and engagement of epitopes on two opposite faces of each RBD might explain the relatively decreased neutralization potency. Overall, the structures support the design of “camelid-like” human Nbs.

### Three Different SARS-CoV-2 RBD Epitopes Targeted by Engineered Nbs

The x-ray structures of Nb225, Nb240, or Nb255 in complex with SARS-CoV-2 RBD revealed that these Nbs bind to the CR3022 cryptic epitope site in an approach angle similar to YYDRxG antibodies (e.g. COVA1-16 and ADI-62113) as reported recently (36, 37) (FIG. 5C). The CDR3 of these Nbs is mainly responsible for the binding and contributes to around 94% of the buried surface area on the RBD. The structural comparison showed that CDR3 of Nb225, Nb240, and Nb255 forms similar hairpin structures as the YYDRxG (SEQ ID NO: 9) motif albeit these Nbs use different sequences compared to YYDRxG Abs (FIGS. 5C, 17). Although Nb225, Nb240, and Nb255 did not compete with ACE2 binding due to their smaller size comparing to COVA1-16 and other YYDRxG Abs (FIG. 12), combination of these Nbs with LM18 or Nb136 demonstrated broad neutralization against SARS-CoV-2 variants including omicron (FIG. 4A-C). None of the publicly available human Ab sequences have a similar sequence as CDR3 in these Nbs, suggesting the engineering approach described herein expanded the structural constraints of Ab binding to SARS-CoV-2 RBD.

Using focused classification and local refinement methods on a single RBD in the cryo-EM data (FIG. 5D, E), an atomic model was generated and found that LM18 binds RBS-D epitope that is located between the CR3022 cryptic site and the RBS-A epitope (e.g. CC12.1) in a manner that is predicted to sterically clash with and compete with Abs CR3022 and CC12.1 (FIGS. 5E,F). Similar to the CR3022 cryptic epitope Nbs above, approximately 88% of the buried surface area is contributed by CDR3. Nb136 binds on the opposite face of the RBD in a region that would sterically clash with RBS-B (e.g. CC6.30) and RBS-C (e.g. C104) Abs (FIGS. 5E, G). Unlike the other Nbs, two CDRs are involved with binding to RBD, with CDR2 and CDR3 contributing approximately 30% and 50%, respectively, to the buried surface area. The epitopes of all 5 Nbs, as determined by high resolution structural analysis, validates the FACS-based epitope binning strategy used for selection and engineering.

Construction of library. The library diversity in the CDR3 loop was generated using primers synthesized with trimer phosphoramidite pools encoding amino acids in the CDR3 hypervariable region at the frequencies observed in naturally occurring Abs and Nbs. Methionines and cysteines were excluded from this mixture to avoid producing Nbs with unwanted chemical liabilities. Full length DNA encoding the human-based Nbs was generated by combining the reverse translated hV_{H}323 scaffold with the CDR3 primers that encoded the diverse CDR3 loops and the invariant framework 4 domain.

In total, CDR3 loop lengths were produced ranging from 10 to 20 amino acids. The frequency of these loops was expressed using a sigmoidal distribution rather than mimicking the naturally occurring normal distribution to avoid oversampling the shorter loops in the library described herein (FIG. 7). This library was transformed into yeast via homologous recombination with an estimated diversity of 3×10⁹ unique clones, based on colony forming assays from dilutions following the transformation.

Magnetic-activated cell sorting (MACS) of naïve library. For the first MACS step, 5.10¹⁰ induced cells were sorted using 50 nM of biotinylated SARS-CoV-2 RBD as the antigen. The binders to streptavidin MicroBeads (Miltenyi Biotec) pre-coupled with the antigen were selected, then grown and induced to be sorted against the streptavidin MicroBeads alone in the absence of the antigen. For this negative selection step, the streptavidin binders that have been enriched from the first round of MACS were discarded and the non-binders were sorted again with Anti-Biotin MicroBeads (Miltenyi Biotec) pre-coupled with 50 nM of biotinylated SARS-CoV-2 RBD. The selected cells were then grown and induced again. These three MACS steps are thought to have decreased the diversity of the library to ~10⁷ different clones (38).

Fluorescence-activated cell sorting (FACS) after MACS steps. FACS is used after depleting the library of non-binding clones by MACS to enrich the yeast cells in RBD-specific clones. In vitro engineering of Abs or Nbs can lead to constructs that are polyspecific (39), so positive and negative selections were alternated to enhance the specificity of the synthetic constructs. The cells were alternatively selected as RBD binders (affinity sorts), or as poly-specificity reagents (PSR) non-binders (negative sorts). In each round of selection, 1-5.10⁷ induced yeast cells were incubated for 60 min at 4 °C (rotating at 50 rpm) with biotinylated SARS-CoV-2 RBD for affinity sorts, or biotinylated HEK-cell soluble membrane protein extracts (40) for PSR sorts in 500 μl 1% PBSA (PBS containing 1% BSA) or PBS, respectively. Yeast cells were then washed twice with 1% PBSA (affinity sorts) or PBS (PSR sorts) and coupled to 2 fluorophores (1 μg/mL) for 20 min: anti-V5-AF405-conjugated to check the yeast display, and anti-biotin-APC or streptavidin-PE to check RBD binding (the use of biotin-specific secondary Ab was alternated to prevent the enrichment in secondary Ab/fluorophore specific clones). Yeast cells were then washed once with 1% PBSA and resuspended in 1 mL 1% PBSA for sorting on a FACS Melody (BD Biosciences). Selected yeast cells were sorted into SD-Ura medium, grown and induced for consecutive rounds of selection. For AFF1, AFF2 and AFF3, 100, 20 and 4 nM of biotinylated SARS-CoV-2 RBD were used, respectively. For PSR1 and PSR2, 10 μg of biotinylated CHO-cell soluble membrane protein extracts were used. Sorted cells were either prepared for NGS sequencing, or serial dilutions of the last affinity sorts were plated on SD-Ura agar. After 3 days at 30 °C, DNA of single colonies was amplified using Phire Plant PCR kit (Thermo Fisher) and sent for Sanger sequencing.

hNb323 sequencing and analysis. Libraries were deep sequenced to determine the CDR3 at each round of selection. The DNA from the sorted yeast cells was miniprepped (Qiagen) in the presence of zymolyase (Zymo Research) and amplified through two rounds of PCR. The first PCR reaction generates a ~200 bp amplicon containing flanking universal Nextera sequencing adapters. The second round of PCR adds specific barcodes so the library can be pooled, cleaned, and sent for deep sequencing on an Illumina MiSeq (Illumina Incorporated, San Diego CA) with the paired-end MiSeq v2 500 bp kit.

Scaffold optimization. Prior to affinity-maturation, it was first determined if the scaffold of LM18 could be improved. A hundred VHH sequences from camelids found in the PDB were overlaid and 11 positions were selected to be rationally mutated (M34, G35, Y37, E44, R45, W47, V48, A50, I51, S74 and L78 according to Kabat numbering) into the commonly found residues in those V_{HH}s. This mini library of ~6000 variants was transformed into yeast cells together with LM18 CDR3 and the linear vector. 5 rounds of FACS (AFF1-PSR1-AFF2-PSR2-AFF3) ere then performed, selecting the best displaying/best binding population, based on the reasoning that a better display correlates with a more stable construct. NGS analysis of the AFF3 sort revealed that the initial residues (MGYERWVAISL (SEQ ID NO: 7)) were preferred, except for position 34, where Leu (77%) is favored compared to Met (23%), and position 79, which could equally be Ile, Val or Leu instead of Leu, validating the robustness of LM18 scaffold. Nevertheless, the M34L mutation was incorporated for further designs of LM18.

NGS analysis of the on-yeast epitope binning. Only sequences counted at least 10 times for at least 1 sort were taken into account. First, it was made sure that sequences that were not specific for RBD (highly enriched sequences from other sorts loaded in the same MySeq run sometimes appear in non-related sorts) were discarded (column “RBD specific”, n). Then, sequences were selected that were “truly enriched” (a “true enrichment” was defined as follow: sequences being present in more than 1 sort with no sequencing mistake, meaning with more than 1 amino acid difference; column “true enrichment”, y). A total of 123 unique CDR3s were obtained. Epitope bins were assigned by defining an overlapping epitope with a tested SARS-CoV-2 Ab as having a C/NC (compete/noncompete) ratio>10, and NC/C ratio>10 for a nonoverlapping epitope. 13 CDR3s were identified in the NC bin of the 3 Abs, while 4 CDR3s were identified in the C bin of the 3 Abs.

Affinity-maturation of LM18. To select high-affinity Nb variants, a new library was prepared that consists of five ultramers and three CDR oligo pools containing a single mutation compared to the original LM18 sequence. The libraries contain 4.2×10⁶ variants (138 CDR1s, 116 CDR2s and 261 CDR3s). Linear pYDSI2SI_Di-U vector was transformed into the YVH10 strain of S. cerevisiae, together with the PCR-amplified DNA library. Four rounds of FACS-based selection (AFF1-AFF2-PSR1-AFF3) was performed to isolate populations of high-affinity clones. Serial dilutions of the AFF3 sort were plated on SD-Ura agar. After 3 days at 30 °C, DNA of 96 single colonies was amplified using Phire Plant PCR kit (Thermo Fisher) and sent for Sanger sequencing.

Protein expression and purification. All recombinant soluble proteins from SARS-CoV, SARS-CoV-2 and their truncated protein versions (RBD) were expressed and purified as previously described (41).

Nbs and Abs expression and purification. Nbs-Fc and Abs (HC and LC constructs) were transiently expressed with the Expi293 Expression System (Thermo Fisher). After five days, 24-deep well culture supernatants were harvested and purified using protein A magnetic beads (Thermo Fisher) and tested for binding and neutralization. Selected Nbs and Abs were re-expressed in small to medium scale cultures and IgG-purified on Protein A sepharose (GE Healthcare). Constructs were buffer exchanged in PBS and stored at 4 °C. His₁₀-tagged Nbs used for SPR and crystallization were purified with the HisPur Ni-NTA Resin (Thermo Fisher). To eliminate nonspecific binding proteins, each column was washed with at least 3 bed volumes of wash buffer (25 mM Imidazole in TBS, pH 7.4). To elute the purified proteins from the column, five bed volumes of elution buffer (250 mM Imidazole in TBS, pH 7.4) was used. Constructs were buffer exchanged in TBS and stored at room temperature.

Recombinant Protein ELISAs. His₆-tagged mAb (Invitrogen, MA1-21315-1MG) was coated onto high-binding 96-well plates (Corning, 3690) at 2 μg/mL overnight at 4 °C. After washing, plates were blocked with 3% BSA in PBS for 1 h. Then His₁₀-tagged recombinant RBD or spike protein were captured at 1 μg/mL in 1% BSA and incubated for 1 h at RT. After washing, serially diluted Nbs or Abs were added into wells and incubated for 1 h at RT. Detection was measured with alkaline phosphatase-conjugated goat anti-human IgG Fcγ (Jackson ImmunoResearch 109-005-008) at 1:1000 dilution for 1 h. After the final wash, phosphatase substrate (Sigma-Aldrich, S0942-200TAB) was added into wells. Absorption was measured at 405 nm after less than 15 min. Positive and negative controls were systematically used. Non-linear regression curves were plotted using Prism 8 software.

Pseudovirus (PSV) Assay. PSV assays were performed according to the protocol described by Rogers et. al. (41)

Epitope binning by bio-layer interferometry (BLI). Nbs were binned into epitope specificities using an Octet RED384 system. 50 nM of His₁₀-tagged RBD protein antigen were captured using anti-Penta-HIS biosensors (18-5120, Molecular Devices). After RBD loading for 5 min, a saturating concentration of mAbs (CR3022, CC6.30 or CC12.1), LM18-Fc or ACE2 (100 μg/mL) was added until saturation. Competing concentrations of Nbs (25 μg/mL) were then added for 5 min to measure binding in the presence of saturating mAbs (or LM18 or ACE2). All incubation steps were performed in 1× PBST (PBS with 0.1% TWEEN 20).

Surface Plasmon Resonance (SPR) Methods. SPR measurements were collected using a Biacore 8K instrument at 25 °C. All experiments were carried out with a flow rate of 30 μL/min in a mobile phase of HBS-EP+ [0.01 M HEPES (pH 7.4), 0.15 M NaCl, 3 mM EDTA, 0.0005% (v/v) Surfactant P20]. Two chips were prepared in order to obtain data for the Nbs (Histagged) and Nbs-Fc and bispecifics. One, anti-Human IgG (Fc) antibody (Cytiva) was immobilized to a density of ~2000-4000 RU via standard NHS/EDC coupling to a Series S CM-3 (Cytiva) sensor chip; a reference surface was generated through the same method. Two, recombinant CoV2-RBD was immobilized to a density of ~250 RU via standard NHS/EDC coupling to flow cell 2 of a Series S CM-5 (Cytiva) sensor chip; a reference surface was generated through activation/deactivation of flow cell 1.

For conventional kinetic/dose-response, bsNb₄-Igs were captured to ~50-100 RU via Fc-capture on the active flow cell prior to analyte injection. A concentration series of CoV2-RBD or CoV2-Spike were injected across the antibody and control surface for 2 min, followed by a 20 min dissociation phase using a multi-cycle method. Regeneration of the surface in between injections of antigen was achieved by two, 120 s injections of 3M MgCl₂ . For conventional kinetics/dose-response of the Nbs, a CoV2-RBD sensor chip was prepared as stated above. A concentration series of each Nb was injected over CoV2-RBD and a control surface for 3 min, followed by a 15 min dissociation phase using a multi-cycle method. Regeneration of the surface in between injections of nanobody was achieved with a single, 60 s injection of 10 mM glycine (pH 1.5), 200 mM NaCl. Kinetic analysis of each reference subtracted injection series was performed using the BIAEvaluation software (Cytiva). Sensorgrams were fit to either a 1:1 (Langmuir) binding or Heterogeneous ligand model.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

### REFERENCES

1. W. T. Harvey, et al., SARS-CoV-2 variants, spike mutations and immune escape. Nat. Rev. Microbiol. 19, 409-424 (2021).
2. A. Baum, et al., Antibody cocktail to SARS-CoV-2 spike protein prevents rapid mutational escape seen with individual antibodies. Science 369, 1014-1018 (2020).
3. E. De Genst, et al., Molecular basis for the preferential cleft recognition by dromedary heavy-chain antibodies. Proc. Natl. Acad. Sci. U.S.A. 103, 4586-4591 (2006).
4. R. A. Weiss, C. T. Verrips, Nanobodies that Neutralize HIV. Vaccines (Basel) 7 (2019).
5. V. Bobkov, et al., Nanobody-Fc constructs targeting chemokine receptor CXCR4 potently inhibit signaling and CXCR4-mediated HIV-entry and induce antibody effector functions. Biochem. Pharmacol. 158, 413-424 (2018).
6. N. S. Laursen, et al., Universal protection against influenza infection by a multidomain antibody to influenza hemagglutinin. Science 362, 598-602 (2018).
7. A. Hultberg, et al., Llama-Derived Single Domain Antibodies to Build Multivalent, Superpotent and Broadened Neutralizing Anti-Viral Molecules. PLoS ONE 6, e17665 (2011).
8. L. Detalle, et al., Generation and Characterization of ALX-0171, a Potent Novel Therapeutic Nanobody for the Treatment of Respiratory Syncytial Virus Infection. Antimicrob. Agents Chemother. 60, 6-13 (2016).
9. M. Schoof, et al., An ultrapotent synthetic nanobody neutralizes SARS-CoV-2 by stabilizing inactive Spike. Science 370, 1473-1479 (2020).
10. D. Wrapp, et al., Structural Basis for Potent Neutralization of Betacoronaviruses by Single-Domain Camelid Antibodies. Cell 181, 1004-1015.e15 (2020).
11. A. W. Tarr, et al., An alpaca nanobody inhibits hepatitis C virus entry and cell-to-cell transmission. Hepatology 58, 932-939 (2013).
12. S. Terryn, et al., Protective effect of different anti-rabies virus VHH constructs against rabies disease in mice. PLoS One 9, e109367 (2014).
13. P.-A. Koenig, et al., Structure-guided multivalent nanobodies block SARS-CoV-2 infection and suppress mutational escape. Science 371 (2021).
14. J.-P. Salvador, L. Vilaplana, M.-P. Marco, Nanobody: outstanding features for diagnostic and therapeutic applications. Anal. Bioanal. Chem. 411, 1703-1713 (2019).
15. V. K. Nguyen, R. Hamers, L. Wyns, S. Muyldermans, Camel heavy-chain antibodies: diverse germline V(H)H and specific mechanisms enlarge the antigen-binding repertoire. EMBO J. 19, 921-930 (2000).
16. S. Muyldermans, Nanobodies: natural single-domain antibodies. Annu. Rev. Biochem. 82, 775-797 (2013).
17. C. Vincke, et al., General strategy to humanize a camelid single-domain antibody and identification of a universal humanized nanobody scaffold. J. Biol. Chem. 284, 3273-3284 (2009).
18. L. Riechmann, S. Muyldermans, Single domain antibodies: comparison of camel VH and camelised human VH domains. J. Immunol. Methods 231, 25-38 (1999).
19. I. Zimmermann, et al., Synthetic single domain antibodies for the conformational trapping of membrane proteins. Elife 7 (2018).
20. Y. Hagihara, S. Mine, K. Uegaki, Stabilization of an immunoglobulin fold domain by an engineered disulfide bond at the buried hydrophobic region. J. Biol. Chem. 282, 36489-36495 (2007).
21. B. Briney, A. Inderbitzin, C. Joyce, D. R. Burton, Commonality despite exceptional diversity in the baseline human antibody repertoire. Nature 566, 393-397 (2019).
22. Z. Tu, et al., Landscape of variable domain of heavy-chain-only antibody repertoire from alpaca. Immunology 161, 53-65 (2020).
23. B. M. Chan, A. Badh, K. A. Berry, S. A. Grauer, C. T. King, Flow Cytometry-Based Epitope Binning Using Competitive Binding Profiles for the Characterization of Monoclonal Antibodies against Cellular and Soluble Protein Targets. SLAS Discov 23, 613-623 (2018).
24. E. P. Hudson, M. Uhlen, J. Rockberg, Multiplex epitope mapping using bacterial surface display reveals both linear and conformational epitopes. Sci. Rep. 2, 706 (2012).
25. J. Wang, et al., A Human Bi-specific Antibody against Zika Virus with High Therapeutic Potential. Cell 171, 229-241.e15 (2017).
26. S. Y. Shi, et al., A biparatopic agonistic antibody that mimics fibroblast growth factor 21 ligand activity. J. Biol. Chem. 293, 5909-5919 (2018).
27. A. Ljungars, et al., A bispecific IgG format containing four independent antigen binding sites. Sci. Rep. 10, 1546 (2020).
28. K. M. Müller, K. M. Arndt, W. Strittmatter, A. Plückthun, The first constant domain (C_{H}1 and C_{L}) of an antibody used as heterodimerization domain for bispecific miniantibodies. FEBS Letters 422, 259-264 (1998).
29. L. Pekar, et al., Biophysical and biochemical characterization of a VHH-based IgG-like bi- and trispecific antibody platform. MAbs 12, 1812210 (2020).
30. U. Brinkmann, R. E. Kontermann, The making of bispecific antibodies. mAbs 9, 182-212 (2017).
31. M. Yuan, et al., A broad and potent neutralization epitope in SARS-related coronaviruses. Proc. Natl. Acad. Sci. U.S.A. 119, e2205784119 (2022).
32. A. Z. Wec, et al., Broad neutralization of SARS-related viruses by human monoclonal antibodies. Science 369, 731-736 (2020).
33. T. F. Rogers, et al., Isolation of potent SARS-CoV-2 neutralizing antibodies and protection from disease in a small animal model. Science 369, 956-963 (2020).
34. R. Yan, et al., Structural basis for the different states of the spike protein of SARS-CoV-2 in complex with ACE2. Cell Res. 31, 717-719 (2021).
35. F. Zhao, et al., Broadening a SARS-CoV-1 neutralizing antibody for potent SARS-CoV-2 neutralization through directed evolution https:/doi.org/10.1101/2021.05.29.443900.
36. H. Liu, et al., Cross-Neutralization of a SARS-CoV-2 Antibody to a Functionally Conserved Site Is Mediated by Avidity. Immunity 53, 1272-1280.e5 (2020).
37. H. Liu, et al., A recurring YYDRxG pattern in broadly neutralizing antibodies to a conserved site on SARS-CoV-2, variants of concern, and related viruses https:/doi.org/10.1101/2021.12.15.472864.
38. C. McMahon, et al., Yeast surface display platform for rapid discovery of conformationally selective nanobodies. Nat. Struct. Mol. Biol. 25, 289-296 (2018).
39. S. A. Sievers, L. Scharf, A. P. West Jr, P. J. Bjorkman, Antibody engineering for increased potency, breadth and half-life. Curr. Opin. HIV AIDS 10, 151-159 (2015).
40. Y. Xu, et al., Addressing polyspecificity of antibodies selected from an in vitro yeast presentation system: a FACS-based, high-throughput selection and analytical tool. Protein Eng. Des. Sel. 26, 663-670 (2013).
41. T. F. Rogers, et al., Isolation of potent SARS-CoV-2 neutralizing antibodies and protection from disease in a small animal model. Science 369, 956-963 (2020).

What is claimed is:

1. A VH domain comprising:
a) one or more of a tyrosine at position 37 (Y37), glutamate at position 44 (E44), and arginine at position 45 (R45);
b) cysteines at positions 49 and 69 (C49 and C69), wherein the cysteines are capable of forming a disulfide bond; and
c) one or more of a glutamine at position 5 (Q5), glycine at position 35 (G35), lysine at position 83 (K83), proline at position 84 (P84), tyrosine at position 102 (Y102) and glutamine at position 108 (Q108),
wherein the VH domain is capable of specifically binding to a target of interest, and wherein the amino acid positions are according to Kabat.

2. The VH domain of claim 1 comprising Y37, E44 and R45.

3. The VH domain of claim 1 or claim 2 comprising Q3, G35, K83, P84, Y102 and Q108.

4. The VH domain of any one of claims 1 to 3 further comprising a CDR3 region comprising 5 to 20 amino acid residues.

5. The VH domain of any one of claims 1 to 4, wherein the FW3-CDR3-FW4 region comprises the amino acid sequence of 92-CARX(5 to 15)FDYW-103, wherein X(5 to 15) stands for 5 to 15 amino acid residues.

6. The VH domain of any one of claims 1 to 5 comprising one or more of
a) the amino acid sequence of EVQLQESGGGLVQPGGSLRLSCAASGFTFS (SEQ ID NO: 1) comprising 0, 1, 2, 3, 4 or 5 substitutions;
b) the amino acid sequence of WYRQAPGKEREWVC (SEQ ID NO: 2) comprising 0, 1, 2, 3, 4 or 5 substitutions;
c) the amino acid sequence of RFTCSRDNSKNTLYLQMNSLKPEDTAVYYCAX (SEQ ID NO: 3) comprising 0, 1, 2, 3, 4 or 5 substitutions; and
d) the amino acid sequence of WGQGTQVTVSS (SEQ ID NO: 4) comprising 0, 1, 2, 3, 4 or 5 substitutions.

7. The VH domain of any one of claims 1 to 6 comprising
a) the amino acid sequence of EVQLQESGGGLVQPGGSLRLSCAASGFTFS (SEQ ID NO: 1) comprising 0, 1, 2, 3, 4 or 5 substitutions;
b) the amino acid sequence of WYRQAPGKEREWVC (SEQ ID NO: 2) comprising 0, 1, 2, 3, 4 or 5 substitutions;
c) the amino acid sequence of RFTCSRDNSKNTLYLQMNSLKPEDTAVYYCAX (SEQ ID NO: 3) comprising 0, 1, 2, 3, 4 or 5 substitutions; and
d) the amino acid sequence of WGQGTQVTVSS (SEQ ID NO: 4) comprising 0, 1, 2, 3, 4 or 5 substitutions.

8. The VH domain of any one of claims 1 to 7 comprising
a) the amino acid sequence of EVQLQESGGGLVQPGGSLRLSCAASGFTFS (SEQ ID NO: 1);
b) the amino acid sequence of WYRQAPGKEREWVC (SEQ ID NO: 2);
c) the amino acid sequence of RFTCSRDNSKNTLYLQMNSLKPEDTAVYYCAX (SEQ ID NO: 3); and
d) the amino acid sequence of WGQGTQVTVSS (SEQ ID NO: 4).

9. The VH domain of any one of claims 1 to 7 comprising
a) an amino acid sequence having at least 80%, 85%, 90%, 95%, 97%, 97%, 98% 99% or 100% identity to EVQLQESGGGLVQPGGSLRLSCAASGFTFSSYAMGWYRQAPGKEREWVCAISGS GGSTYYADSVKGRFTCSRDNSKNTLYLQMNSLKPEDTAVYYCA (SEQ ID NO: 5); and
b) the amino acid sequence of WGQGTQVTVSS (SEQ ID NO: 4) comprising 0, 1, 2, 3, 4 or 5 substitutions.

10. The VH domain of any one of claims 1 to 7 comprising
a) the amino acid sequence of EVQLQESGGGLVQPGGSLRLSCAASGFTFSSYAMGWYRQAPGKEREWVCAISGS GGSTYYADSVKGRFTCSRDNSKNTLYLQMNSLKPEDTAVYYCA (SEQ ID NO: 5); and
b) the amino acid sequence of WGQGTQVTVSS (SEQ ID NO: 4).

11. The VH domain of any one of claims 1 to 7 comprising
a) an amino acid sequence having at least 80%, 85%, 90%, 95%, 97%, 97%, 98% 99% or 100% identity to EVQLQESGGGLVQPGGSLRLSCAASGFTFSSYAMGWYRQAPGKEREWVCAISGS GGSTYYADSVKGRFTCSRDNSKNTLYLQMNSLKPEDTAVYYCA (SEQ ID NO: 5); and
b) the amino acid sequence of DYWGQGTQVTVSS (SEQ ID NO: 6) comprising 0, 1, 2, 3, 4 or 5 substitutions.

12. The VH domain of any one of claims 1 to 7 comprising
a) the amino acid sequence of EVQLQESGGGLVQPGGSLRLSCAASGFTFSSYAMGWYRQAPGKEREWVCAISGS GGSTYYADSVKGRFTCSRDNSKNTLYLQMNSLKPEDTAVYYCA (SEQ ID NO: 5); and
b) the amino acid sequence of DYWGQGTQVTVSS (SEQ ID NO: 6).

13. The VH domain of any one of claims 1 to 12, wherein the VH domain comprises a VH3_23 human framework.

14. The VH domain of any one of claims 1 to 13, wherein the target of interest is a pathogen derived antigen.

15. The VH domain of any one of claims 1 to 14, wherein the target of interest is a pathogen derived polypeptide.

16. The VH domain of claim 15, wherein the pathogen derived polypeptide comprises a viral, bacterial, or parasitic polypeptide.

17. The VH domain of claim 15, wherein the pathogen derived polypeptide comprises a viral polypeptide.

18. The VH domain of claim 17, wherein the viral polypeptide comprises a Betacoronavirus, Chikungunya virus, Dengue virus, Ebola virus, Eastern Equine Encephalitis virus, Herpes Simplex virus, Human Cytomegalovirus, Human Papillomavirus. Human Metapneumovirus, Influenza virus, Japanese Encephalitis virus, Marburg virus, Measles, Parainfluenza virus, Respiratory Syncytial virus, Sindbis virus, Varicella Zoster virus, Venezuelan Equine Encephalitis virus, West Nile virus, Yellow Fever virus, or Zika virus polypeptide or an immunogenic fragment thereof.

19. The VH domain of claim 15, wherein the viral polypeptide comprises a MERS-CoV polypeptide, SARS-CoV polypeptide, SARS-CoV-2 polypeptide or an immunogenic fragment thereof.

20. The VH domain of claim 15, wherein the viral polypeptide comprises a SARS-CoV polypeptide, SARS-CoV-2 polypeptide or an immunogenic fragment thereof.

21. The VH domain of claim 15, wherein the viral polypeptide comprises a SARS-CoV-2 spike protein (S), SARS-CoV-2 envelope protein (E), SARS-CoV-2 nucleocapsid protein (N), SARS-CoV-2 membrane protein (M) or an immunogenic fragment thereof.

22. The VH domain of claim 15, wherein the viral polypeptide comprises a SARS-CoV-2 spike protein (S) or an immunogenic fragment thereof.

23. The VH domain of any one of claims 1 to 13, wherein the target of interest is a tumor antigen.

24. A multispecific binding agent comprising at least two VH domains according to any one of claims 1 to 23, wherein the at least first VH domain and at least second VH domain binds to different epitopes.

25. The multispecific binding agent of claim 24, wherein the at least first VH domain and at least second VH domain binds to different epitopes on the same antigen.

26. The multispecific binding agent of claim 24, wherein the at least first VH domain and at least second VH domain binds to different antigens.

27. The multispecific binding agent of any one of claims 24 to 26, which is a bispecific agent.

28. The multispecific binding agent of any one of claims 24 to 26, which is a trispecific agent.

29. A fusion polypeptide comprising at least one VH domain according to any one of claims 1 to 23 and a heterologous polypeptide.

30. The fusion polypeptide of claim 29 comprises more than one VH domain.

31. The fusion polypeptide of claim 29 or claim 30, wherein the heterologous polypeptide is
a) an antibody,
b) an antibody fragment,
c) an Fc domain,
d) transmembrane domain, or
e) a membrane associating domain.

32. The fusion polypeptide of claim 31, wherein the heterologous polypeptide is an Fc domain.

33. The fusion polypeptide of claim 32, wherein the Fc domain is a modified Fc domain.

34. The fusion polypeptide of claim 32 or claim 33, wherein the Fc domain is a human Fc domain.

35. The fusion polypeptide of any of claims 32 to 34, wherein the Fc domain comprises a modification that promotes heterodimer formation.

36. The fusion polypeptide of claim 35, wherein the Fc domain comprises a knob mutation or a hole mutation.

37. A chimeric antigen receptor (CAR) comprising a VH domain according to any one of claims 1 to 23.

38. A host cell comprising the CAR of claim 37.

39. The host cell of claim 38, which is a T cell or NK cell.

40. A polynucleotide encoding the VH domain, multispecific binding agent, fusion polypeptide or CAR according to any one of the preceding claims.

41. The polynucleotide of claim 40, which is a DNA or RNA.

42. The polynucleotide of claim 40, which is an mRNA comprising a modification.

43. A vector comprising the polynucleotide of claim 40.

44. The vector of claim 43, which is a viral vector.

45. A host cell comprising the polynucleotide of any one of claims 40 to 42, or the vector of claim 43 or claim 44.

46. The host cell of claim 45, which is a T cell or NK cell, wherein the polynucleotide encodes a CAR.

47. A method of neutralizing a pathogen in a subject in need thereof, comprising administering to the subject a composition comprising a VH domain of any of claims 1 to 23, or a polynucleotide encoding the VH domain.

48. A method of treating a subject in need thereof, comprising administering to the subject a composition comprising a VH domain of any of claims 1 to 23, or a polynucleotide encoding the VH domain.

49. A VH domain library comprising a plurality of VH domains according to any one of claims 1 to 23.

50. A polynucleotide library comprising a plurality of polynucleotides encoding VH domains according to any one of claims 1 to 23.

51. A method for identifying a VH domain capable of binding a target of interest, comprising
a) contacting the VH domain library of claim 49 with the target of interest, and
b) identifying a VH domain capable of binding to the target of interest.

52. A method for identifying a polynucleotide encoding a VH domain capable of binding a target of interest, comprising
a) expressing the polynucleotide library of claim 50 in a suitable host cell to produce a library of cells expressing a VH domain;
b) contacting the library of cells with the target of interest, and
c) identifying a polynucleotide encoding a VH domain capable of binding to the target of interest.

53. A vector comprising
a) at least one functional heavy chain V gene,
b) at least one heavy chain D gene and at least one heavy chain J gene, and
c) a murine C gene which lacks the CH1 exon,
wherein the at least one functional heavy chain V gene encodes a polypeptide comprising a fragment of the VH domain according to any one of claims 1 to 12, wherein the fragment consists of the residues corresponding to the human VH3_23 gene.

54. The vector of claim 53, wherein the at least one heavy chain D gene is a human D gene and the at least one heavy chain J gene is a human J gene.

55. The vector of claim 53, wherein the at least one heavy chain D gene is a murin D gene and the at least one heavy chain J gene is a murine J gene.

56. The vector of any one of claims 53 to 55, wherein the at least one functional heavy chain V gene encodes a polypeptide comprising the amino acid sequences of claim 7 a), b), and c).

57. The vector of any one of claims 53 to 55, wherein the at least one functional heavy chain V gene encodes a polypeptide comprising the amino acid sequences of claim 8 a), b), and c).

58. The vector of any one of claims 53 to 55, wherein the at least one functional heavy chain V gene encodes a polypeptide comprising the amino acid sequences of claim 9 a).

59. The vector of any one of claims 53 to 55, wherein the at least one functional heavy chain V gene encodes a polypeptide comprising the amino acid sequences of claim 10 a).

60. The vector of any one of claims 53 to 59, further comprising sufficient regulatory sequence elements to enable the at least one functional heavy chain V gene, at least one heavy chain D gene, at least one heavy chain J gene and murine C gene to undergo rearrangement to produce a functional VHH antibody.

61. A transgenic mouse host cell transformed with the vector of any one of claims 53 to 60.

62. A transgenic mouse comprising the vector of any one of claims 53 to 60 or the cell of claim 61.

63. The transgenic mouse of claim 62, wherein said mouse comprises one or more of
a) a non-functional endogenous lambda light chain locus,
b) a non-functional endogenous kappa light chain locus, and
c) non-functional endogenous heavy chain locus.

64. The use of the transgenic mouse of claim 62 or 63 or the transgenic mouse host cell of claim 62 in producing a VH only antibody or a VH domain that is capable of specifically binding to a target of interest.

65. The use of the transgenic mouse of claim 62 or 63 in constructing a VH domain library.

66. The use of the transgenic mouse of claim 62 or 63 in constructing a naïve VH domain library.
